(19) United States
(12) Patent Application Publication
(10) Pub. No.: US 2010/0035992 A1
(43) Pub. Date: Feb. 11, 2010

(54) Methods and Compositions for Treating Inflammation and Inflammation-Related Pathologies

(76) Inventors: Rajiv Bhushan, (US); Chris Duffield, (US); Jerry G. Gin, (US); Doug Gjerde, (US); Amit Goswamy, (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/402,484

(22) Filed: Mar. 11, 2009

Related U.S. Application Data
(60) Provisional application No. 61/035,706, filed on Mar. 11, 2008.

(51) Int. Cl. A61K 31/195; A61P 29/00; A61P 1/00; A61P 13/12

(52) U.S. Cl. 514/566

(57) ABSTRACT

Methods and compositions are provided for the treatment of inflammation and disorders, diseases, and adverse conditions, i.e., pathologies, caused by or otherwise associated with inflammatory processes. A metal ion sequestering agent that directly or indirectly exerts an anti-inflammatory effect is administered to a subject in combination with a sequestration inactivating moiety that facilitates transport of the metal ion sequestering agent through biological membranes. The sequestration inactivating moiety also inactivates the metal ion sequestering agent until association between the two components is cleaved in vivo to release the active sequestering agent. Compositions containing a metal ion sequestering agent and a sequestration inactivating moiety are also provided; the compositions optionally contain an added anti-inflammatory agent.

### CROSS-REFERENCE TO RELATED APPLICATION

This application claims priority under 35 U.S.C. §119(e)(1) to provisional U.S. Patent Application Ser. No. 61/035,706, filed Mar. 11, 2008, the disclosure of which is incorporated by reference herein.

### TECHNICAL FIELD

This disclosure relates generally to the field of pharmacotherapy, and more particularly relates to methods and compositions for the prevention and treatment of inflammation and conditions associated with inflammation. The disclosure finds utility in the fields of medicine, pharmacology, and drug delivery.

### BACKGROUND

Inflammation is a complex biological response of vascular tissue to harmful stimuli, such as oxidative stress, irritants, pathogens, and damaged cells. It is a protective attempt by the organism to remove an injurious stimulus and initiate the healing process for injured tissue. The inflammatory response involves the production and release of inflammatory modulators that function to both destroy damaged cells and heal injured tissue. In order to perform this function, however, various inflammatory modulators either directly produce and/or signal the release of agents that produce reactive oxygen species for the purpose of destroying invading agents and/or injured cells. The inflammatory response, therefore, involves a balance between the destruction of damaged cells and the healing of injured tissue, since an imbalance can lead to oxidative stress and the onset of various inflammatory disease pathologies.

More specifically, oxidative stress in a biological system is caused by the imbalance between the system's production of reactive oxygen species and the system's actual ability to detoxify and repair the damage resulting from such species. Typical formulations for the prevention and/or treatment of oxidative stress involve the administration of antioxidants, i.e., agents that primarily function by reducing the rate at which oxidation occurs or otherwise inhibiting the oxidation of other compounds. Many antioxidants involve a post-oxidation mechanism in which free radical chain reactions initiated by free radicals produced during oxidation are terminated. Other antioxidants work by undergoing direct oxidation by free radicals, thus reducing the fraction of other compounds that are oxidized.

The use of such antioxidants to reduce oxidative stress and/or prevent or treat disease is, however, controversial. Further, although the administration of antioxidants may function to slow or prevent the oxidation of various compounds in the body, they typically do not function to treat and/or prevent the underlying mechanisms that lead to oxidative stress. More specifically, with respect to the present disclosure, typical antioxidants do not function to prevent and/or treat inflammation, which often involves or leads to oxidative stress.

Accordingly, there is a need in the art for methods and compositions that not only prevent and/or treat inflammation but also reduce oxidative stress and/or prevent and/or treat inflammation-related pathologies. The subject methods and compositions presented herein meet these and other needs in the art.

### SUMMARY OF THE DISCLOSURE

In one aspect of the disclosure, a method is provided for treating an inflammatory condition in a subject. The method involves administering to the subject an effective amount of an inactivated metal ion sequestering agent that is readily transported through biological membranes and which is activated in vivo to sequester metal ions that are directly causing, indirectly causing, or otherwise associated with the inflammatory condition. The metal ion sequestering agent is in inactivated form prior to administration. For instance, the metal ion sequestering agent may be in inactivated form by virtue of being associated with an effective amount of a sequestration inactivating moiety that inactivates the ability of the metal ion sequestering agent to sequester metal ions. The sequestration inactivating moiety may also facilitate transport of the metal ion sequestering agent through biological membranes. The inactivated metal ion sequestering agent is sometimes referred to herein as a “prochelator,” although sequestration of metal ions can involve sequestration and complexation processes beyond the scope of chelation per se. The term “prochelator” is analogous to the term “prodrug” insofar as a prodrug is a therapeutically inactive agent until activated in vivo, and the prochelator, as well, is incapable of sequestering metal ions until activated in vivo. The use of prochelator components and compositions in the treatment of inflammatory conditions, as described herein, is believed to be a completely novel and unprecedented discovery.

The metal ion sequestering agent and the sequestration inactivating moiety are generally, although not necessarily, administered in a single composition in which the two components are combined. In such a case, there may be some fraction of each component that is not associated with the other, but the majority of each component will be associated with the other as explained herein. The method may also involve separate administration of the metal ion sequestering agent and the sequestration inactivating moiety, or, in some cases, the two components may be incorporated in separate and discrete sections of a dosage form. Accordingly, in another embodiment, a method of the disclosure involves co-administration of a therapeutically effective amount of the metal ion sequestering agent and an amount of a sequestration inactivating moiety effective to inactivate the sequestering agent and facilitate transport thereof through biological membranes.

In another aspect of the disclosure, a composition is provided for the treatment of inflammatory conditions. The composition contains a therapeutically effective amount of an anti-inflammatory agent, a therapeutically effective amount of a metal ion sequestering agent, and, in association with the metal ion sequestering agent, a sequestration inactivating moiety that facilitates the transport of the metal ion sequestering agent through biological membranes, wherein the sequestration inactivating moiety is released in vivo to provide an activated metal ion sequestering agent. The amount of the sequestration inactivating moiety in the composition is sufficient to inactivate the ability of the metal ion sequestering agent to sequester metal ions until the sequestration inactivating moiety is released in vivo.

In a further aspect of the disclosure, an anti-inflammatory composition is provided that consists essentially of a therapeutically effective amount of a metal ion sequestering agent and a sequestration inactivating moiety that is effective facilitate transport of the metal ion sequestering agent through biological membranes, wherein the amount of the sequestration inactivating moiety in the composition is sufficient to inactivate the ability of the metal ion sequestering agent to sequester metal ions until the sequestration inactivating moiety is released in vivo to provide an active metal ion sequestering agent that directly or indirectly exerts an anti-inflammatory effect within the body.

Other features and advantages of the disclosure will be apparent from the following detailed description and claims.

### BRIEF DESCRIPTION OF THE FIGURES

According to common practice, the various features of the drawings may not be presented to-scale. Rather, the dimensions of the various features may be arbitrarily expanded or reduced for clarity. Included in the drawings are the following figures:

FIG. 1 sets forth an illustration of various exemplary and different mechanism of action for a Sequestration Inactivating Moiety+Metal Complexer composition of the disclosure. FIG. 1A depicts the functioning of a composition of the disclosure, including a metal complexer, such as EDTA, and a sequestration inactivating moiety, such as MSM, so as to sequester extra- or intracellular metal ions. FIG. 1B depicts the functioning of a composition of the disclosure including a metal complexer and a sequestration inactivating moiety, such as EDTA and MSM in the prevention of membrane fluidity by sequestering metal ions, such as Fe²⁺ or Fe³⁺, that are essential for the conversion of arachidonic acid to 4-HNE. FIG. 1C depicts the functioning of a composition of the disclosure, including a metal complexer, such as EDTA, and a sequestration inactivating moiety, such as MSM, so as to directly or indirectly activate the production of aldehyde dehyrdogenase 1 (ALDH1), which ALDH1 may prevent the production of 4-HNE. FIG. 1D depicts the functioning of a composition of the disclosure, including a metal complexer, such as EDTA, and a sequestration inactivating moiety, such as MSM, for the modulation of a variety intracellular pathways.

FIG. 2 depicts a micrograph of paraffin rat spleen after 6 hours of saline only treatment, saline+LPS treatment, and MSM+EDTA treatment for the immunohistochemical analysis for TNF-α.

FIG. 3 depicts a micrograph of paraffin-embedded rat spleen after 6 hours of saline only treatment, saline+LPS treatment, and MSM+EDTA treatment for the immunohistochemical analysis for Caspase-3.

FIG. 4 depicts a bar graph illustrating serum IL-6 levels.

FIG. 5 depicts a low magnification photomicrograph of a pancreatic lobule.

FIG. 6 depicts a high magnification photomicrograph of pancreatic endocrine islets.

FIG. 7 depicts photomicrographs of immunostained tissue samples of the eye with respect to staining produced by labeled Anti-NFκB (FIG. 7A), Anti-protein HNE (FIG. 7B), Anti-MMP9 (FIG. 7C), and anti-TNFα antibodies (FIG. 7D).

### DETAILED DESCRIPTION OF THE DISCLOSURE

### Definitions and Terminology

It is to be understood that unless otherwise indicated this disclosure is not limited to particular embodiments described, as such may vary. It is also to be understood that the terminology used herein is for the purpose of describing particular embodiments only, and is not intended to be limiting. Unless defined otherwise, all technical and scientific terms used herein have the same meaning as commonly understood by one skilled in the art to which this disclosure belongs.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Throughout this application, various publications, patents and published patent applications are cited. The disclosures of these publications, patents and published patent applications referenced in this application are hereby incorporated by reference in their entireties into the present disclosure. Citation herein of a publication, patent, or published patent application is not an admission the publication, patent, or published patent application is prior art.

As used herein and in the appended claims, the singular forms “a”, “and”, and “the” include plural referents unless the context clearly dictates otherwise. Thus, for example, “a metal ion sequestering agent” encompasses a plurality of metal ion sequestering agents as well as a single such agent, and reference to “a sequestration inactivating moiety” includes reference to two or more sequestration inactivating moieties as well as a single sequestration moiety, and so forth. It is further noted that the claims may be drafted to exclude any optional element. As such, this statement is intended to serve as antecedent basis for use of such exclusive terminology as “solely”, “only” and the like, in connection with the recitation of claim elements, or the use of a “negative” limitation.

In this specification and in the claims that follow, reference will be made to a number of terms, which shall be defined to have the following meanings:

“Optional” or “optionally present”—as in an “optional additive” or an “optionally present additive” means that the subsequently described component (e.g., additive) may or may not be present, so that the description includes instances where the component is present and instances where it is not.

By “pharmaceutically acceptable” is meant a material that is not biologically or otherwise undesirable, e.g., the material may be incorporated into a formulation of the disclosure without causing any undesirable biological effects or interacting in a deleterious manner with any of the other components of the dosage form formulation. However, when the term “pharmaceutically acceptable” is used to refer to a pharmaceutical excipient, it is implied that the excipient has met the required standards of toxicological and manufacturing testing and/or that it is included on the Inactive Ingredient Guide prepared by the U.S. Food and Drug Administration. As explained in further detail infra, “pharmacologically active” (or simply “active”) as in a “pharmacologically active” derivative or analog refers to derivative or analog having the same type of pharmacological activity as the parent agent.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of an undesirable condition or damage. Thus, for example, “treating” a subject involves prevention of an adverse condition in a susceptible individual as well as treatment of a clinically symptomatic individual by inhibiting or causing regression of the condition.

The term “beneficial agent” refers to any chemical compound, complex or composition that exhibits a desirable effect, e.g., an effect deemed to be beneficial. For instance, in certain embodiments, a beneficial agent may be an agent the administration of which results in a beneficial effect, e.g., a therapeutic effect in the treatment of an adverse physiological condition such as inflammation and inflammation-related pathologies. In certain embodiments, a beneficial agent is one that interacts with the other components of a formulation or dosage form so as to produce a desirable effect. For instance, a beneficial agent may be an agent that affects a formulation of the disclosure in a beneficial way. In certain embodiments, the term may also encompass an agent that interacts with a body, or a body component, to produce a beneficial condition, for example, a reduction in inflammation. Metal ion sequestering agents herein are beneficial agents by virtue of their having a direct or indirect benefit with respect to inflammation, i.e., they are directly or indirectly acting inflammatory agents.

With respect to pharmacologically active agents, the term “beneficial agent” also includes pharmacologically acceptable derivatives of those beneficial agents specifically mentioned herein, including, but not limited to, salts, esters, amides, prodrugs, active metabolites, isomers, analogs, crystalline forms, hydrates, and the like. In certain embodiments, when the term “beneficial agent” is used, or when a particular beneficial agent is specifically identified, it is to be understood that pharmaceutically acceptable, pharmacologically active salts, esters, amides, prodrugs, active metabolites, isomers, analogs, etc. of the beneficial agent are intended as well as the beneficial agent per se. However, it is also to be understood that in certain embodiments, a beneficial agent need not be a pharmacologically active agent or have a therapeutic effect so long as the effect it does have is deemed beneficial, and in some instances, at least neutral, or, if negative, balanced by corresponding benefits.

By an “effective” amount or a “therapeutically effective amount” of a beneficial agent is meant a nontoxic but sufficient amount of the agent to provide the beneficial effect. The amount of beneficial agent that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Unless otherwise indicated, the disclosure is not limited to specific formulation components, modes of administration, beneficial agents, manufacturing processes, or the like, as such may vary.

### Inflammation and Inflammatory Conditions:

The present disclosure provides methods and formulations for the treatment of inflammation and inflammation-related conditions, where “treatment” of such conditions encompasses prevention of such conditions, as noted earlier herein. Inflammation is a complex biological response designed to destroy or inactivate invading pathogens, remove cellular waste and debris, and facilitate restoration of normal function, either through resolution or repair, in response to threatened pathology. In the absence of inflammation, infections, wounds, and irritants would never be healed or removed and progressive destruction of the tissue would result, thereby compromising the survival of the organism.

Inflammation has two main phases: cellular and exudative. The cellular phase involves the extravasation or movement of white blood cells, e.g., leukocytes, out of the blood vessels and toward the site of injury. The exudative phase involves the additional movement of fluid, containing proteins and immunoglobulins, into the inflamed tissue. During both of these phases, blood vessels are dilated upstream and constricted downstream of the injured tissue. Additionally, capillary permeability to the affected site is increased, which results in a net loss of blood plasma into the tissue, giving rise to edema or swelling. Such swelling distends the tissues, compresses nerve endings, and thus causes pain.

The two phases of inflammation are controlled largely by soluble mediators. These soluble mediators regulate the activation of both the resident cells (such as fibroblasts, endothelial cells, tissue macrophages, and mast cells) as well as the newly recruited inflammatory cells (such as monocytes, lymphocytes, neutrophils, and eosinophils) by initiating a plurality of biochemical cascades. These cascades function to recruit leukocytes and/or monocytes, via the increased expression of cellular adhesion molecules and chemoattraction, as well as to propagate and mature the inflammatory response. These cascades include the complement, coagulation, and fibrinolysis systems. Specifically, in response to cellular modulators released by injured tissues, the blood vessels react so as to become more permeable and thereby permit the extravasation of leukocytes through the blood vessel membranes.

Inflammation may either be acute or chronic, depending upon its duration. Generally, acute inflammation is mediated by granulocytes or polymorphonuclear leukocytes, and chronic inflammation is mediated by mononuclear cells, such as monocytes and macrophages.

Acute inflammation is the initial response of the body to harmful stimuli. It is a short-term process that is achieved by the increased movement of plasma and leukocytes, such as granulocytes, and antibodies, from within the blood vessels and into the inflamed tissue surrounding a site of injury. The extravasation and accumulation of plasma and leukocytes into the injured tissue results in the telltale signs of inflammation, including: swelling, redness, heat, pain, and loss of function.

Accordingly, leukocytes play an important role in the initiation and maintenance of acute inflammation by extravasating from the capillaries into injured tissue; acting as phagocytes, picking up bacteria and cellular debris; and walling off infection thereby preventing its spread. Once in the tissue, leukocytes migrate along a chemotactic gradient to reach the site of injury, where they become activated, and attempt to remove the pathological stimulus and effectuate repair of the tissue.

Leukocytes function, in part, by releasing inflammatory cytokines. Generally, the inflammatory cytokines released stimulate neutrophils to enhance oxidative (e.g., superoxide and secondary products) and nonoxidative (e.g., myeloperoxidase and other enzymes) inflammatory activity. For instance, the release of inflammatory cytokines, such as tumor necrosis factor-alpha (TNF-α), is a means by which the immune system combats pathology. Specifically, TNF-α stimulates the expression and activation of adherence factors on leukocytes and endothelial cells, primes neutrophils for an enhanced inflammatory response to secondary stimuli, and enhances adherent neutrophil oxidative activity. Hence, primed neutrophils are characteristic of inflammation as they are one of the first groups of cells to appear in an infected area, and perform many important functions, including phagocytosis and the releasing of inflammatory chemical messengers.

In addition, various leukocytes can be further stimulated to maintain inflammation through the action of an adaptive cascade involving lymphocytes. For instance, lymphocytes, such as T cells, B cells, mast cells, and antibodies, become activated by the presentation of processed antigens displayed on the cell surface of macrophages and dendritic cells. Activation of the aforementioned species in turn stimulates the lymphocytes to act as pro-inflammatory cytotoxic cells. Additionally, activated mast cells release histamine and prostaglandins, while activated macrophages release TNF-α and IL-1. In this manner, acute inflammation may be converted to chronic inflammation.

There are four main consequences of acute inflammation. The first is resolution, which is the complete reconstitution of damaged tissue. Healing, however, in many circumstances, does not occur completely and a scar will form. Hence, the second response involves connective tissue scarring, in which connective tissue is formed so as to bridge any gaps caused by injury. Connective tissue scarring also involves angiogenesis, thereby forming new blood vessels to provide nutrients to the newly formed tissue. For example, after laceration to the skin, a connective tissue scar results which does not contain any specialized structures such as hair or sweat glands. The third and fourth responses involve abscess formation and ongoing or chronic inflammation. An acute inflammatory response continues for as long as the injurious stimulus is present and ceases once the stimulus has been removed, broken down, or walled off by scarring (fibrosis). If the injurious stimulus remains, however, or if the inflammatory response thereto persists, acute inflammation may be converted to chronic inflammation.

Chronic inflammation is prolonged and is characterized by a dominating presence of macrophages in the injured tissue, which extravasate via the same methods discussed above. Macrophages are powerful defensive agents of the body, but the toxins they release (including reactive oxygen species) are injurious to the organism's own tissues as well as to invading agents. Therefore, chronic inflammation is almost always accompanied by tissue destruction. Hence, inflammation involves the simultaneous destruction and healing of the tissue during the inflammatory process.

As the inflammation process shifts from acute to chronic, there is a corresponding and progressive shift in the types of immune cells that are present at the site of inflammation. For instance, neutrophils last for only a short period of time. If the inflammation persists for an extended time period, neutrophils are gradually replaced by longer lasting monocytes. Hence, chronically inflamed tissue is characterized by the infiltration of mononuclear immune cells (monocytes, macrophages, lymphocytes, and plasma cells) into the tissue. These cells function both to destroy and heal the damaged tissue, extra-cellular structures, and surrounding vasculature. Although monocytes collect slowly at inflammatory foci, they develop into long-term resident accessory cells and macrophages. Upon stimulation with an inflammation trigger, monocytes and macrophages produce and secrete an array of cytokines (including TNF-α), complement, lipids, reactive oxygen species, proteases and growth factors that remodel tissue and regulate surrounding tissue functions.

As can be seen with respect to the above, during both the acute and chronic inflammatory processes, and in response to cellular pathology, injured tissues release a host of soluble cellular mediators, such as plasma-derived inflammatory mediators, that affect the cells surrounding a site of injury and activate various inflammatory agents thereby. The cells associated with inflammation include: the vascular endothelium; vascular smooth muscle cells; fibroblasts; myocytes; leukocytes, including neutrophils, eosinophils, lymphocytes, monocytes, and basophils; macrophages; dendritic cells; mast cells, and the like. Such cells further release soluble inflammatory mediators, such as cytokines, that further function to mature and/or prolong the inflammatory immune response.

Hence, although acute inflammation in and of itself may be a normal homeostatic immune response, it involves the release of soluble mediators that initiate biochemical cascades that make the surrounding vasculature more permeable to plasma and leukocytes and create a chemotactic gradient through which those agents may reach a site of injury. The soluble mediators modulating the process of extravasation largely include various cytokines and chemokines. The dysregulation of these cytokines and chemokines can lead to serious inflammatory complications and secondary disease. For instance, the inappropriate and excessive release of inflammatory cytokines, such as TNF-α, IL-1, and/or IL-6, can produce counterproductive exaggerated pathogenic effects through the release of tissue-damaging oxidative and non-oxidative products.

Chronic inflammation as well often leads to ongoing inflammatory complications and system damage. For instance, as the inflammation process shifts from acute to chronic and the types of immune cells present at the site of inflammation correspondingly shift from granulocytes and antibodies to monocytes, macrophages, and lymphocytes, such as natural killer cells and helper T cells, there is a concomitant change in the cellular factors present in the extra-cellular milieu.

For example, as described above, a fundamental component of the chronic inflammatory response mediated by various lymphocytes, such as helper T-cells, entails the cellular release of inflammatory cytokines and a diverse array of cellular mediators. However, the prolonged production of such cellular factors may cause irreparable damage and/or disease to one or more bodily systems if not properly regulated. Specifically, for instance, the over-production of cytokines and cellular mediators such as matrix metalloproteases, TNF-α, TNF-β, interleukins, EGF, bFGF, etc., may lead to tissue destruction, such as that found in many inflammatory conditions. For example, TNF-α can induce neutrophils to adhere to the blood vessel wall and then migrate through the vessel to the site of injury, where it then releases oxidative and non-oxidative inflammatory products, such as reactive oxygen species, that are harmful to both the injured and non-injured cells surrounding the site of injury.

Accordingly, an examination of the mechanisms underlying both acute and chronic inflammation reveals the conflicting processes inherent in inflammation. Removal of harmful stimuli often involves the production of compounds, such as reactive oxygen species, which are toxic to the body. Hence, if left unchecked, inflammation leads to a pathological cycle of destruction and healing of the tissue, which increases the oxidative stress of the entire body system.

The unabated production of reactive oxygen species, which include free radicals and peroxides, for instance, from inflammatory mediators activated during an inflammatory response, is a particularly destructive aspect of oxidative stress. For instance, reactive oxygen species and the like, such as superoxide, are released by macrophages and can be converted, by oxidoreduction reactions with transition metals or other redox cycling compounds including quinones, e.g., in the extracellular milieu, into aggressive radical species, such as hydroxyl radicals, that can cause extensive cellular damage. Most reactive oxygen-derived species are produced at a low level by normal aerobic metabolism and during normal inflammatory responses, and the damage they cause to cells is constantly repaired. Under severe levels of oxidative stress, however, such as is the case in conditions of extreme acute or chronic inflammation, the damage may cause ATP depletion, leading to controlled apoptotic death, and in severe cases necrosis.

Oxidative stress in a biological system is caused by the imbalance between the system's production of reactive oxygen species (and intermediates thereof) to treat a pathological condition, and the system's ability to detoxify and repair the damage resulting from such species. On one hand, the production of reactive oxygen species can be beneficial; for example, reactive oxygen species are employed in some cell signaling processes, termed redox signaling. On the other hand, the overproduction of reactive oxygen species, such as in extreme acute or chronic inflammation, may result in cellular or tissue injury, thereby producing oxidative stress within the system, and potentiating or leading to many families of diseases, as described herein below.

The effects of oxidative stress depend upon the nature and extent of these imbalances. For instance, an un-injured cell is typically able to overcome small perturbations and regain its original state. However, more severe oxidative stress, such as that induced by chronic inflammation, can cause cell death, and even moderate oxidation can trigger apoptosis, while more intense stresses may cause necrosis.

To maintain proper cellular homeostasis, then, especially with respect to the inflammatory response, a balance must be struck in the injured tissue between reactive oxygen production and destruction. For instance, with respect to an individual cell of a tissue, the body functions, in part, to maintain a reducing environment within the cell. Such a reducing environment is preserved by enzymes that maintain the reduced state through a constant input of metabolic energy. Injury to the cell causes disturbances in the normal redox state of the cell. Because of the body's inflammatory response, which results as an attempt to heal injured cells, such disturbances may have toxic effects for the tissue surrounding an injured cell. For instance, in an attempt to heal the injured site, various inflammatory agents may be released and/or recruited, and which may then trigger the production of peroxides and free radicals that can cause damage to surrounding cells, including the proteins, lipids, and DNA therein, causing cell death and thereby increasing the oxidative stress in the overall body system.

These disturbances in the normal redox state of the cell, for instance, induced by an unfettered immune response, may be caused by several mechanisms. For instance, the extra-cellular generation of electron donors, such as peroxide or superoxide, e.g., during an inflammatory response, may interact with metal ions in the extracellular milieu so as to generate highly reactive extra-cellular oxidants. For example, iron, including the ferric iron (Fe²⁺) and the ferrous ion (Fe³⁺), in the interstitial fluid or plasma may react with oxygen, superoxide, or peroxide produced by inflammatory mediators and/or immune cells, such as in a Fenton/Haber Weiss reaction, to initiate a chain reaction that results in the production of highly reactive hydroxyl radicals, which in turn may damage surrounding cells and exacerbate oxidative stress.

For instance, the extracellular oxidants produced, such as hydroxyl radicals, may directly damage the phospholipid components of the cell walls of surrounding cells in the tissue. That is, extracellular oxidants, such as those produced by cytokines as described above, may directly interact, in a free radical chain reaction, with polyunsaturated fatty acids in the cell membrane to produce lipid radicals and lipid peroxy radicals that may further react to produce lipid peroxides. This lipid peroxidation reaction may lead to the oxidative degradation of the lipids, which in turn results in direct damage to the cell walls.

Further, a main byproduct of the lipid peroxidation reaction is the generation of 4-hydroxynonenal (4-HNE). 4-HNE is a very reactive unsaturated hydroxyalkenal that interacts with proteins in the cell membrane to produce protein aggregates. This aggregation of proteins within the cell membrane may also damage the cell wall.

Furthermore, 4-HNE may be produced by the direct interaction of extracellular oxidants with arachidonic acid present in the phospholipids of cell membranes. Arachidonic acid is a polyunsaturated fatty acid that may react with extracellular oxidants so as to produce cytotoxic lipid-derived aldehydes. Specifically, arachidonic acid may interact with oxidants produced in an inflammatory response to generate 11-hydroperoxide. The hydroperoxide produced may then react with Fe²⁺ and Fe³⁺ to generate 4-HNE.

Accordingly, extracellular oxidants may adversely affect the membranes of cells by directly damaging the phospholipids within the cell membrane and/or by initiating a chain reaction that produces 4-HNE, which in turn damages the cell membrane. The damage to the cell membrane makes the cell membrane more permeable to extracellular ionic species, such as calcium (Ca²⁺), potassium (K⁺), Fe²⁺, Fe³⁺ and the like. This is problematic because when the cell membrane becomes more permeable to charged species, such as Ca²⁺, K⁺, Fe²⁺, Fe³⁺, and the like, such ions are free to enter the cell along their concentration gradient, which results in an abnormally high concentration of such ions in the cell. At high concentrations within the cell, these metallic cations may function as secondary messengers initiating deleterious cascades that result in further damage and even death to the cell, e.g., via apoptosis or necrosis, as well as damage to the surrounding tissue.

For instance, high levels of intracellular calcium may initiate a plurality of cascades, such as the caspase and/or protein kinase C(PKC) cascades, which may result in further damage to the cell and/or surrounding tissues. That is, at high concentrations, both Ca²⁺ and 4-HNE may act as intracellular modulators that are capable of triggering toxic cell death pathways. Specifically, both Ca²⁺ and 4-HNE are capable of inducing cysteine-aspartic acid proteases (“caspase”) enzymes, thereby provoking the cleavage of various substrates in the cell, such as lamin and poly(ADP-ribose) polymerase (“PARP”), which in turn results in cell death. 4-HNE may also cause the laddering of genomic DNA and/or the release of cytochrome c from mitochondria.

Calcium ions may also induce PKC, which in turn may activate transforming growth factor β-activated kinase 1 (“TAK1”). TAK1 may then activate one or both of the mitogen-activated protein kinase (MAPK) and the IκB kinase (IKK) cascades, which cascades may initiate AP-1 and NF-κB transcription, both of which lead to the increased production of inflammatory cytokines such as TNFα, interleukin-1 (IL-1), interleukin-6 (IL-6), interferon (IFN), monocyte chemotactic protein (MCP), matrix metalloproteinases (MMPs), and the like. The production and release of these and other cytokines from the cell may then initiate or produce an acute or chronic inflammatory response resulting in the increased production of reactive oxygen species and subsequent increased oxidative stress, which in turn, as explained above, may lead to the increased induction of pathways that trigger inflammation, which inflammation may be amplified and run unchecked.

Inflammation that runs unchecked is thus very problematic and can lead to a host of diseases and other adverse physiological conditions. Regardless of the type of inflammation, chronic or acute, inflammatory processes underlie pathologies affecting a wide variety of organ systems. For instance, inflammatory mediators and cytokines, such as those described above, e.g., TNF-α, Il-1 and Il-6, have been shown to be pathogenic in various circumstances in their propensity to produce reactive oxygen species which, if left unchecked, lead to oxidative stress. Unabated inflammation plays a role in many disease pathologies including but not limited to: hypersensitivities; immune and autoimmune related diseases; gastrointestinal diseases; various types of cancer; vascular complications; heart diseases; neurodegenerative diseases; kidney related diseases; reproductive inflammatory disease, including pelvic inflammatory disease; vasculitis; chronic prostatitis; gout; ulcer-related diseases; age related diseases; preeclampsia; diseases related to chemical, radiation, or thermal trauma; and other inflammatory diseases as will be recognized by those of ordinary skill in the art and/or described in the pertinent literature and texts.

For instance, inflammatory complications have been found to be involved with several different hypersensitivities. One group of hypersensitivity-related maladies encompasses allergic diseases such as asthma, hay fever, rhinitis, vernal conjunctivitis, and other eosinophil-mediated conditions. For instance, asthma is a disease with two major components, a marked inflammatory reaction, and a disorder involving bronchial smooth muscle reactivity that results in bronchospasms. Increased production of inflammatory mediators causes infiltration of leukocytes, such as lymphocytes, eosinophils, and mast cells, into the tissues of the lungs, thereby producing oxidative stress and inflammation.

Both oxidative stress and inflammation in the lungs and/or gastrointestinal tract can lead to increased complications in individuals afflicted with cystic fibrosis. For instance, the blockage of airways due to the overproduction of mucus and/or phlegm that occurs with cystic fibrosis may be exacerbated by inflammatory conditions and/or conditions of oxidative stress. This exacerbation may in turn lead to tissue injury and/or structural damage within the linings of the lungs. The resultant tissue and/or structural damage may lead to chronic breathing problems.

Other types of inflammatory allergic diseases include rhinitis, conjunctivitis, and urticaria. In all of these allergic diseases, a multiplicity of allergens triggers the infiltration and activation of “allergic” classes of leukocytes, e.g., eosinophils, mast cells and basophils, resulting in the subsequent release of histamine, platelet activating factor, etc., thereby causing inflammation and oxidative stress.

Additional hypersensitivity-related maladies are adverse skin reactions such as psoriasis, contact dermatitis, eczema, infectious skin ulcers, open wounds, cellulitis, and the like. Psoriasis, for example, is a chronic inflammatory skin disorder involving hyperproliferation of the epidermis and inflammation of both the epidermis and the dermis. In psoriasis, macrophage and neutrophil infiltration of the dermis and epidermis is seen, and proinflammatory mediators are released from the activated cells, in turn producing inflammation and oxidative stress.

Additionally, inflammatory complications have been found to be associated with a host of immune and autoimmune disorders. Such disorders include, by way of example, arthritis, myopathies, types I and II diabetes, gastrointestinal diseases, transplant rejection, and the like. Inflammation-related arthritic disorders include, for instance, rheumatoid arthritis, osteoarthritis, spondyloarthropathies, myopathies, and the like. In rheumatoid arthritis, the synovial tissue lining a joint forms a mass that infiltrates and degrades articular cartilage, tendons, and bone. Normal synovial tissue consists of a thin membrane of two or three cell layers that include fibroblast-like synovial cells and rare resident macrophages. In contrast, rheumatoid synovial tissue consists of a mixture of cell types: immune T- and B-cells, monocyte/macrophages, polymorphonuclear leucocytes, and fibroblast-like cells, which have a rampant proliferative ability. Most of these cells are recruited to the rheumatoid joint in response to inflammatory stimuli that occur as part of the pathology of this disease, and thus, their presence initiates a cytotoxic cascade the results in increased oxidative stress.

Although the etiology of rheumatoid arthritis is not clear, it is suspected that an antigen such as a bacterium, virus, or mycoplasma, is deposited in the joints as a consequence of a systemic infection. Normally, the antigen would be cleared and no disease arises. However, in genetically or otherwise susceptible individuals, the antigen elicits an acute inflammatory response in which autologous tissue damage occurs. This, in turn, produces an (auto)immune response, which eventually leads to a chronic inflammatory and immunologic reaction within the synovial lining of the joint and oxidative stress. Thus, there is a plurality of activated cell types, and the cytokines the activated cells produce continuously fuel the proliferative and destructive ability of the synovial fibroblasts, leading to rheumatoid arthritis.

Osteoarthritis (also known as degenerative joint disease) involves gradual breakdown of cartilage and is usually but not always associated with aging. There are two types of osteoarthritis (OA): primary and secondary OA. Primary osteoarthritis, is caused by cartilage damage resulting from increasing stress on a joint, e.g., from obesity. In primary OA, the articular cartilage of the joint is slowly roughened over time, which roughening is followed by pitting, ulceration, and progressive loss of cartilage surface. Secondary OA is caused by trauma or chronic joint injury due to some other type of arthritis, such as rheumatoid arthritis, or from overuse of a particular joint. Although most body tissues can make repairs following an injury, in primary and secondary OA, cartilage repair is hampered by a limited blood supply and the lack of an effective mechanism for cartilage re-growth, and yet the presence of inflammatory cytokines (such as IL-1, TNF-α, and metalloproteases) within the joint area are increased. Accordingly, in both types of OA, degenerative changes to the articular cartilage, subchondral bone, and the synovial membrane occur after the joints are subjected to repeated damage (mechanical or otherwise) and prolonged inflammation.

Another type of inflammation-induced arthritic disorders are the spondyloarthropathies. The diseases classified as spondyloarthropathy are psoriatic arthritis (PsA), juvenile chronic arthritis with late pannus onset, enterogemic spondyloarthropathies (enterogenic reactive arthritis (ReA) and inflammatory bowel diseases (IBD)), urogenital spondyloarthropathies (urogenital ReA), and undifferentiated spondyloarthropathies. In spondyloarthropathy arthridity, various types of immune-mediated joint inflammation produce degenerative changes in the joints. The changes consist of infiltration of inflammatory intermediaries, such as IL-1, TNF-α, and metalloproteases, within the synovial membranes as well as degeneration of the articular cartilage and associated subchondral bone.

Moreover, there are many common myopathies that are not technically classified as arthritis, but involve similar symptoms, are due to injury, strain, and inflammation of tendons or ligaments, the latter condition sometimes referred to as “soft tissue rheumatism.” Some of the more common soft tissue rheumatic conditions include tennis elbow, frozen shoulder, carpal tunnel syndrome, plantar fasciitis, and Achilles tendonitis. Tennis elbow is due to inflammation of the tendons of the hand gripping muscles where these tendons ultimately attach to the elbow. Frozen shoulder is a stiffening of the ligaments around the shoulder joint, and is usually induced by swelling and inflammation. Carpal tunnel syndrome involves a nerve which passes through the carpal tunnel on the front of the wrist into the human hand. When this nerve becomes inflamed it presses on the walls of the tunnel causing pain. Plantar fasciitis involves ligaments in the sole of the foot that become inflamed, resulting in pain in the foot, and tends to occur in individuals who stand for long periods of time throughout the day. Spurs, such as calcium spurs in the heels or joints, may be the product of both inflammation and overproduction of calcium, whereby calcium deposits form on the bone. Achilles tendonitis involves inflammation of the Achilles tendon, causing pain while walking. Other myopathies include acute muscle and soft-tissue injury, as well as vascular insufficiency that leads to edema, such as lower leg edema.

Type I diabetes, is also an inflammation-induced disease, and is generally classified as a T cell-mediated chronic autoimmune disease. It involves the generation of an inflammatory immune response that results in the destruction of pancreatic islets. Specifically, cells associated with inflammatory processes such as lymphocytes and TNF-α, infiltrate and attack the pancreatic insulin-producing β-cells in the islets of Langerhans (insulitis). This attack results in the selective destruction of the β-cells, thereby leading to insulin-dependent diabetes mellitus (IDDM), and systemic oxidative stress.

Inflammation-induced autoimmune diseases also include the gastrointestinal disease referred to as “gastrointestinal inflammation.” That disease involves inflammation of a mucosal layer of the gastrointestinal tract (including the upper and lower gastrointestinal tract), and encompasses acute and chronic inflammatory conditions.

Chronic gastrointestinal inflammation includes inflammatory bowel disease, or “IBD,” which refers to any of a variety of diseases characterized by inflammation of all or part of the intestines. Examples of inflammatory bowel disease include, but are not limited to, Crohn's disease, Barrett's syndrome, ileitis, irritable bowel syndrome, irritable colon syndrome, ulcerative colitis, pseudomembranous colitis, hemorrhagic colitis, hemolytic-uremic syndrome colitis, collagenous colitis, ischemic colitis, radiation colitis, drug and chemically induced colitis, diversion colitis, colitis in conditions such as chronic granulomatous disease, celiac disease, celiac sprue, food allergies, gastritis, infectious gastritis or enterocolitis (e.g., Helicobacter pylori-infected chronic active gastritis), pouchitis and other forms of gastrointestinal inflammation caused by an infectious agent, and other like conditions.

IBD is referenced as exemplary of gastrointestinal inflammatory conditions, and is not meant to be limiting. Clinical and experimental evidence suggest that the pathogenesis of IBD is multifactorial and involves the susceptibility of the immune system to adverse environmental factors. The interaction of these factors with the immune system results in a broad range of host reactions including the overproduction of inflammatory mediators, which leads to intestinal inflammation, oxidative stress, and dysregulated mucosal immunity against commensal bacteria, various microbial products, (e.g., LPS) and antigens (Mayer et al. Current concept of IBD: Etiology and pathogenesis in “Inflammatory Bowel Disease,”5th edition 2000, Kirsner J B editor. W.B. Saunders Company, pp 280-296). Accordingly, cytokine imbalance and the production of inflammatory mediators have been postulated to play an important role in the pathogenesis of both colitis and IBD. For instance, animal models of colitis have highlighted the prominent role of CD4+ T cells in the regulation of intestinal inflammation.

Another type of inflammation-induced autoimmune-related disease includes graft-versus-host-disease (GVHD). In GVHD, immunologic recognition and the immune response are caused by histocompatibility differences between the donor and recipient as well as by cytotoxicity caused by alloreactive T cells. For instance, cellular injury in GVHD is caused by cellular infiltration of effector cells into target tissues which results in inflammation and cellular destruction.

Other types of autoimmune-related diseases caused by or associated with inflammation include systemic lupus erythematosus, (SLE), lupus nephritis, Addison's disease, Myasthenia gravis, vasculitis (e.g. Wegener's granulomatosis), autoimmune hepatitis, osteoporosis, and some types of infertility. For instance, osteoporosis, such as postmenopausal osteoporosis, is characterized by a progressive loss of bone tissue, which leads to the occurrence of spontaneous fractures. A mechanism for the onset of osteoporosis involves an increase in the secretion of modulatory factors such as IL-1, IL-6, and TNF-α, and TNF-β, which are produced in the bone microenvironment and influence bone remodeling. Specifically, IL-1 and TNF-α promote bone resorption in vitro and in vivo by activating mature osteoclasts indirectly, via a primary effect on osteoblasts, and by stimulating the proliferation and differentiation of osteoclast precursors. IL-6 also increases osteoclast formation from hemopoietic precursors. Additionally, infertility can involve a disorder of the ovary that results in abnormal folliculogenesis, in which leukocytes infiltrate the follicular fluid and when activated produce inflammatory cytokines such as IL-1, IL-6 and TNF-α.

Inflammatory complications have also been found to be involved with tumor metastases and several different cancers. For instance, the processes of tumor invasion and metastasis depend upon increased proteolytic activity of invading tumor cells. Matrix metalloproteinases, cathepsins B, D, and L, and plasminogen activator participate in this metastatic cascade. Additionally, blood coagulability increases due in part to the oxidative stress caused by cancer and/or heart disease, leading to coagulation problems.

Further still, inflammatory conditions have been found to be involved with various aberrant responses in endothelial tissues, which may result in vascular complications such as vascular inflammatory disease, associated vascular pathologies, atherosclerosis, an giopathy, inflammation-induced atherosclerotic and thromboembolic macroangiopathy, coronary artery disease, as well as cerebrovascular and peripheral vascular diseases. Atherosclerosis, for example, involves the narrowing of a blood vessel lumen due to the production of an atherosclerotic plaque. Such plaques are problematic in that due to increased concentrations of various metalloproteases, derived from inflammatory cells within the plaque, the plaques may rupture and thereby causing embolisms, strokes, and/or a heart attack.

Consequently, inflammatory conditions have been found to be involved with various heart diseases and/or other cardiac complications. Such complications include cardiovascular circulatory diseases induced or exacerbated by an inflammatory response, such as ischemia/reperfusion, atherosclerosis, peripheral vascular disease, restenosis following angioplasty, inflammatory aortic aneurysm, vasculitis, stroke, spinal cord injury, congestive heart failure, hemorrhagic shock, ischaemic heart disease/reperfusion injury, vasospasm following subarachnoid hemorrhage, vasospasm following cerebrovascular accident, pleuritis, pericarditis, inflammation-induced myocarditis, the cardiovascular complications of diabetes, and the like. For instance, ischemia-induced endothelial cell injury provoked by an aberrant inflammatory response has been described as being a pivotal causative event leading to an array of pathophysiologic sequelae, such as microvascular vasoconstriction, adhesion and aggregation of platelets and neutrophils, and deceased blood flow. Specifically, the infiltration and activation of multiple types of inflammatory cells results in a series of degenerative changes in the vasculature of the affected area, which causes damage to the surrounding parenchymal tissue, and leads to ischemia and oxidative stress.

Further, inflammatory conditions have been found to be involved with brain swelling and various neurodegenerative diseases. For instance, multiple sclerosis (MS) is an inflammatory demyelinating disorder of the central nervous system (CNS). MS is characterized histopathologically by focal lesions in different stages of evolution in the white matter of the CNS. Breakdown of the blood-brain barrier and inflammatory perivascular infiltration are the first events in lesion formation and are followed by demyelination and astrogliosis. Local inflammation is induced by an autoimmune response against the myelin sheath, such as when proteolytic enzymes and matrix metalloproteases contribute to inflammatory tissue damage. Specifically, immune abnormalities have been described in the peripheral blood and cerebrospinal fluid of MS patients, including the presence of inflammatory T-cells, increased synthesis of immunoregulatory cytokines, and oligoclonal immunoglobulin.

Inflammatory conditions have also been found to be involved with various kidney related, pancreatic, liver, and pelvic inflammatory diseases and conditions, such as kidney disease, nephritis, glomerulonephritis, dialysis, peritoneal dialysis, pericarditis, chronic prostatitis, vasculitis, gout, and the like. For instance, acute pancreatitis is a severe inflammation of the pancreas that often results in pancreatic necrosis. In the early stages of acute pancreatitis, elevated serum levels of IL-1, IL-6, and TNF-α are frequently seen. Additionally, chronic inflammation may lead to increased iron production and overload, producing liver damage, which in turn may lead to fibrosis and cirrhosis. Conversely, liver damage caused by alcohol, drugs, or hepatitis C may lead to inflammation, which in turn may further increase liver damage. Other iron overload diseases, such as those caused by genetic diseases, may lead to or be exacerbated by inflammation, which, in combination with the iron overload caused by the underlying disease, may lead to the onset of other associated diseases such as liver disease, diabetes, arthritis, and the like.

Additionally, anemia, or at least the complications associated with anemia, may be increased by inflammation and/or oxidative stress. For instance, anemia may be caused by oxidative stress that disrupts iron homeostasis signals and the underlying mechanisms thereof thereby leading to anemia associated complications.

Further still, inflammatory conditions have been found to be involved with various ulcer related diseases, such as peptic ulcer disease, acute pancreatitis, aphthous ulcers, and the like. For instance, peptic ulcers are the result of an imbalance between aggressive (acid, pepsin) and protective (mucus, bicarbonate, blood flow, prostaglandins, etc.) factors. Infection of the mucosa of the human gastric antrum with the bacterium Helicobacter pylori has been widely accepted as a cause of chronic, active, type B gastritis. This form of gastritis has been linked directly to peptic ulcer disease by studies showing that eradication of H. pylori reverses this gastritis and prevents duodenal ulcer relapse. Because cytokines are the principal mediators by which immune/inflammatory cells communicate with each other and with other cells, it is likely that these agents are involved in the pathogenesis of chronic active type B gastritis and the resulting peptic ulcer disease. Additionally, aphthous ulcers are caused by an autoimmune phenomenon that provokes the destruction of discrete areas of the oral mucosa, which leads to oral ulcerations. Among the cytokines present in these active areas of ulceration, TNF-α appears to play a predominant role.

Additionally, inflammatory conditions have been found to be involved with various age-related diseases. For instance, because diseases such as atherosclerosis (plaque rupture), fibrosis, osteoporosis, and many others, are associated with increased levels of inflammatory cytokines, such as IL-1, IL-6 and TNF-α, this suggests that physiological aging in humans is associated with an increased capability of peripheral blood mononuclear cells to produce proinflammatory cytokines. On the other hand, many diseases associated with pre-maturity, for instance, retinopathy, chronic lung disease, arthritis, and digestive problems, may be due in part to iron overload and/or inflammation.

Further, inflammatory conditions have been found to be involved with preeclampsia. Preeclampsia is characterized by development of hypertension, endothelial cell disruption, coagulopathy, leukocyte activation, edema, renal dysfunction, and fetal growth disturbances. The endothelial cell damage seen in preeclampsia is produced in part by TNF-α. In preeclampsia, trophoblast growth and differentiation are abnormal, plasma volume expansion fails to occur and TNF-α levels are elevated.

Furthermore, inflammatory conditions have been found to be involved with chemical or thermal trauma due to burns, acid, and alkali, chemical poisoning (MPTP/concavalin/chemical agent/pesticide poisoning), snake, spider, or other insect bites, adverse effects from drug therapy (including adverse effects from amphotericin B treatment), adverse effects from immunosuppressive therapy, (e.g., interleukin-2 treatment), adverse effects from OKT3 treatment, adverse effects from GM-CSF treatment, adverse effects of cyclosporine treatment, and adverse effects of aminoglycoside treatment, stomatitis and mucositis due to immunosuppression. Inflammation may also result of exposure to ionizing radiation, such as solar ultraviolet exposure, nuclear power plant or bomb exposure, or radiation therapy exposure, such as for therapy for cancer.

Additionally, inflammation and/or oxidative stress may lead to blood lipid alteration resulting in the formation of metal-rich, such as calcium rich, complexed lipid deposits.

Further, inflammation in the dental region may be caused by inflammation that results from gingivitis, periodontitis, and/or physical trauma.

As can be seen with respect to the above, the two stages of inflammation, when precisely regulated, promote the health of body tissues by destroying and repairing injured cells and thereby maintaining the well-being of the body as a whole. In order to perform this function, however, the inflammatory system relies on soluble modulators, such as inflammatory cytokines, both to signal cellular injury and to direct the breakdown and healing of injured cells and tissues.

For instance, as described above, injured cells and tissues release a wide variety of soluble factors and cytokines, including matrix metalloproteases or metalloproteinases (MMPs), TNF-α, TNF-β, interleukins, EGF, bFGF, etc., so as to initiate and maintain an immune response. Once initiated, the acute inflammatory stage involves the recruitment and extavasation of leukocytes to a site of cellular injury within the tissue. Once at a site of injury, the recruited leukocytes both release inflammatory cytokines such as tumor necrosis factor-alpha (TNF-α), IL-1 and/or IL-6, and initiate the lymphocyte cascade that results in the production and attraction of macrophages. Additionally, the chronic inflammatory stage involves the extavasation of monocytes and macrophages to a site of cellular injury within the tissue. When activated macrophages release TNF-α, IL-1, and/or IL-6 all of which stimulate the production of oxidative products (e.g., reactive oxygen species) that not only attack the injured cells, but also attack the cells of the surrounding tissue, and in some instances, even distant tissues, such as secondary inflammatory responses distanced from an initial or primary site of inflammation.

Hence, although the release of inflammatory soluble factors and cytokines, such as MMPs, TNF-α, IL-1, and/or IL-6, is a means by which the immune system combats pathology, the dysregulation of these factors, cytokines, and other modulators of the inflammatory pathways may lead to oxidative stress, which can in turn cause serious inflammatory complications and secondary diseases. The inappropriate overproduction of inflammatory modulators and cytokines can produce counterproductive exaggerated pathogenic effects through the release of tissue-damaging oxidative products, such as reactive oxygen species, including free radicals and peroxides, both of which increase oxidative stress and lead to the disease pathologies described above. Such dysregulation of the immune system may lead to a feedback response or mutual reinforcement cycle during which an increase in an inflammatory response results in an increase in oxidative stress, with the increase in oxidative stress resulting in a further increase in inflammation, etc., and vice versa.

### Methods and Compositions for Treating Inflammation:

Accordingly, in view of the above, the present methods and compositions are directed to the prevention and/or treatment of inflammation and inflammation-related pathologies, by alleviating oxidative stress, reducing and/or preventing the effects of reactive oxygen species, preventing lipid peroxidation, inhibiting and down-regulating the formation of 4-HNE, reducing the intracellular concentration of Ca²⁺, inhibiting the Ca²⁺ caspase and PKC pathways, preventing cell death, reducing production of inflammatory modulators such as TNF-α, IL-1, IL-6, IFN, MCP, MMPs, and the like, and/or inhibiting MMPs, as well as reducing metal (e.g., iron and calcium) loading.

In one embodiment, a method of treating inflammation in a subject involves administration of (1) a therapeutically effective amount a metal ion sequestering agent, e.g., a chelating agent, wherein the metal ion sequestering agent directly or indirectly has a beneficial anti-inflammatory effect, and (2) a sequestration inactivating moiety that acts as a transport enhancing agent and is present in amount effective to inactivate the metal ion sequestering agent. By an amount of the sequestration inactivating moiety “effective to inactivate the sequestering agent” is meant an amount that will inactivate at least 50 wt. % of the metal ion sequestering agent, preferably at least 75% of the sequestering agent, optimally at least 90% of the sequestering agent, and most preferably at least 99 wt. % of the sequestering agent. The metal ion sequestering agent, when in active, or “activated” form, i.e., when not associated with a sequestration inactivating moiety, is capable of sequestering metal ions that are associated with a dysfunctional inflammatory process in some way, e.g., they may act as catalysts in oxidative reactions, e.g., Fe²⁺ and Fe³⁺, in the extracellular milieu, thereby reducing the availability of such reactive metal ions for participating in the production of reactive oxygen species. By sequestering reactive metal ions, both in the extra-cellular milieu and within the cell membrane, the sequestering agents herein are capable of preventing lipid peroxidation reactions, thereby preventing the production of lipid peroxides, and are also capable of inhibiting the conversion of arachidonic acid to 4-HNE. In this manner, a composition of the present disclosure functions to reduce oxidative stress, for instance, by sequestering reactive metal ions that act as catalysts in oxidation reactions, and preventing their participation in the generation of reactive oxygen species, thereby inhibiting lipid peroxidation production and reducing the formation of 4-HNE.

The aforementioned method will generally, although not necessarily, involve administration of the metal ion sequestering agent and the sequestration inactivating moiety in a single composition, such that the sequestering agent is in inactivated form when administered to the patient.

With regard to the sequestration inactivating moiety, specifically, it is to be emphasized that the moiety selected acts not only to inactivate the metal ion sequestering agent, but doubles as a transport enhancing agent, i.e., the agent inactivates the sequestering agent until the agent is activated in vivo, and also facilitates transport of the sequestering agent (in inactivated form) into and through body tissues and membranes, e.g., into and through phosphlipid membranes, into cells, and, in certain instances, into the organelles thereof, such as the nucleus and/or mitochondria. Facilitation of any or all of these processes, in which an agent passes into or through one or more biological membranes, is encompassed by the term “transport enhancement” as used herein. For example, because many chelating agents and other sequestering agents contain negatively charged coordinating atoms (e.g., ionized carboxylic acid groups, or carboxylates), they do not readily penetrate the membranes of cells, but rather are repelled thereby. Accordingly, in certain embodiments, the sequestration inactivating moiety functions, in part, to mask the charge of a sequestering agent, thereby allowing the agent to enter the biological membranes such as cell membranes and/or pass therethrough.

Hence, in certain embodiments, the present disclosure is directed to the transportation of a metal ion sequestering agent, such as a chelating agent, into and/or through a biological membrane such as a cell membrane within which the agent is capable of sequestering reactive metal ions therein, e.g., Ca²⁺, and thereby breaking up metal complexes of lipids and/or proteins, so as to repair, restore normal membrane morphology, and minimize the effects of oxidative stress.

Further, in certain embodiments, the present disclosure is directed to the transportation of a metal ion sequestering agent through the cell membrane and into the cell and, in some instances, into the organelles within the cell. Once in the cell, the metal ion sequestering agent functions to sequester intracellular metal ions, such as Ca²⁺, thereby reducing the intracellular levels thereof. In this manner, a composition of the present disclosure functions to inhibit the Ca²⁺ caspase and PKC pathways.

Specifically, both the caspase and PKC families require high concentrations of Ca²⁺ so as to be activated. By sequestering calcium and inhibiting the activation of these pathways, a composition of the present disclosure functions, to prevent or reduce the caspase-induced apoptotic pathway and prevent or reduce the MAPK and NIK pathways that lead to the increased transcription of AP1 and NF-κB. Accordingly, in at least this manner, a composition of the present disclosure is capable of reducing the production of pro-inflammatory modulators, such as TNF-a, IL-1, IL-6, IFN, MCP, MMPs, and the like, as well as preventing and/or treating inflammation.

Accordingly, a composition of the present disclosure is effective for preventing and/or treating inflammation and thereby is useful for the prevention and treatment of various inflammation-induced pathologies, such as those described herein, for instance, hypersensitivities; immune and autoimmune diseases and disorders; gastrointestinal diseases; various types of cancer; vascular complications; heart diseases; neurodegenerative diseases; kidney related diseases; pelvic inflammatory disease, vasculitis, chronic prostatitis; gout; ulcer-related diseases; age-related diseases and disorders; preeclampsia; diseases related to chemical, radiation, or thermal trauma; and other conditions, disorders and diseases caused by or otherwise associated with acute and/or chronic inflammation.

Additionally, compositions of the present disclosure are effective for detoxifying 4-HNE. 4-HNE is detoxified by reaction with aldehyde dehydrogenase (ALDH). For example, ALDH1 oxidizes 4-HNE and thereby detoxifies 4-HNE. The compositions of the disclosure are effective for up-regulating ALDH and thereby detoxifying 4-HNE. Accordingly, the compositions of the present disclosure are effective for up-regulating ALDH, detoxifying 4-HNE, and thereby preventing the deleterious effects of 4-HNE and the disease pathologies associated therewith.

Further, the composition of the present disclosure are capable of preventing or at least minimizing tissue damage caused by increased deleterious activity of MMPs, for instance, by inactivating MMPs, thereby ameliorating the harmful effects thereof.

The disclosure is not limited with respect to the mechanism and/or linkage by which the sequestration inactivating moiety acts to inhibit the ability of the metal ion sequestering agent to sequester metals. Generally, the sequestration inactivating moiety may be any chemical compound, ion, or molecular fragment that inactivates the ability of the metal ion sequestering agent to sequester metal ions and acts as a transport enhancer, by facilitating transport of the sequestering agent through biological membranes. The association between the metal ion sequestering agent and the sequestration inactivating moiety is cleaved following administration and/or upon reaching a location in the body where a dysfunctional inflammatory process is occurring. Cleavage of the association results in the release of the sequestration inactivating moiety in vivo to provide an activated metal ion sequestering agent, which can then act to sequester metal ions that are directly or indirectly associated with inflammatory processes.

For instance:

(1) The metal ion sequestering agent and the sequestration inactivating moiety may be covalently attached, with the covalent linkage or linkages between the two severed by a chemical reaction in vivo. That reaction may be enzymatic or nonenzymatic, triggered, for instance, by an abundance of hydrogen peroxide at a local site within the body that is experiencing oxidative stress.

(2) The sequestration inactivating moiety may be a cationic species, typically a metal ion, which is chelated, complexed, or otherwise sequestered by the metal ion sequestering agent prior to administration. In this case, the sequestration inactivating moiety, i.e., the cation, is selected so that the cation to be sequestered displaces the cationic sequestration inactivating moiety in vivo but not prior to administration or prior to encountering the cation to be sequestered within the body.

(3) The sequestration inactivating moiety can also ionically bind to one or more coordinating atoms in the metal ion sequestering agent, with the ionic bond cleaving in vivo. For instance, with a metal ion sequestering agent comprising a chelator containing at least one coordinating nitrogen atom, the sequestration inactivating moiety would be an anionic species that associates with the nitrogen atom to form an ion pair, where the anionic species is displaced and the nitrogen atom converted to the electronically neutral state in vivo. With a metal ion sequestering agent that comprises a chelator containing at least one coordinating oxygen atom, e.g., in a carboxylate group, the sequestration inactivating moiety is cationic, associated with the carboxylate group in the form of an ion pair. As with the previous example, the cationic species in association with the oxygen atom prior to administration is displaced and the oxygen atom is converted to the electronically neutral state in vivo.

(4) The sequestration inactivating moiety may also associate with the metal ion sequestering agent via one or more hydrogen bonds, where the sequestration inactivating moiety thus “masks” the coordinating atom or atoms in the metal ion sequestering agent and prevents sequestration until the sequestration inactivating moiety is released in vivo.

(5) The sequestration inactivating moiety may be a charge masking agent of a different type, e.g., it may be an aprotic solvent. Charge masking agents can work in different ways and have various functions, any or all of which improve the activity or effectiveness of the metal ion sequestering agent. Charge masking agents can, for instance, facilitate the passage of the sequestering agent across a membrane or other biological barriers. They may also: facilitate diffusion across and into various biological media and solutions; act upon biological solids to change their structure or nature to allow the sequestering agent to enter or act on the solid or react or otherwise interact with a biological solid; and/or help break down, remove, and/or dissolve solids.

The sequestration inactivating moiety, in each of these systems, should be selected such that it enables transport of the metal ion sequestering agent as explained above. It should have minimal or no toxicity, and, once separated from the metal ion sequestering agent in vivo, its cleavage product or other degradation products should have minimal or no toxicity as well. Ideally, the sequestration inactivating moiety should enable the metal ion sequestering agent to reach its intended target, i.e., the site of oxidative stress and/or inflammation, before releasing the agent as an active sequestering species.

Chelators, ligands, and other species that act as iron sequestering agents include the siderophores desferrioxamine (deferoxamine, DFO, desferrioxamine B, Desferal) and desferrithiocin; desferri-exochelin; 4-[3,5-bis-(hydroxyphenyl)-1,2,4-triazol-1-yl]-benzoic acid (ICL670A); 4′-hydroxydesazadesferrithiocin (4,5-dihydro-2-(2,4-dihydroxyphenyl)-4-methylthiazole-4-carboxylic acid; deferitrin); deferiprone (1,2-dimethyl-3-hydroxypyridin-4-one); hydroxypyridinone analogs; aroylhydrazones such as pyridoxal isonicotinoyl hydrazone and analogs thereof, e.g., 2-pyridylcarboxaldehyde isonicotinoyl hydrazone and its analogs, and di-2-pyridylketone isonicotinoyl hydrazone and its analogs; thiosemicarbazones such as triapine (3-aminopyridine-2-carboxaldehyde thiosemicarbazone); the polyamino carboxylic acid ethylenediamine tetraacetic acid (EDTA) and salts thereof; N,N′-di(2-hydroxybenzyl)ethylenediamine-N,N′-diacetic acid HCl (HBED); deferasirox; hydroxamic acid analogs such as 4-aminophenylhydroxamic acid, 2-aminophenylhydroxamic acid, and salicylhydroxamic acid; rhodotorulic acid; N,N′-bis(2-hydroxybenzyl)prop-ylene-1,3-diamine-N,N-diacetic acid (HBPD), 2,3-dihydroxybenzoic acid; and diethyltriamine pentaacetic acid (DTPA).

Examples of chelators, ligands, and other species that act as calcium sequestering agents include, without limitation, the polyamino carboxylic acids EDTA, ethylene glycol tetraacetic acid (EGTA), 1,2-bis(o-aminophenoxy)ethane-N,N,N′,N′-tetraacetic acid) (BAPTA); and the esterified BAPTA analog 1,2-bis-(o-aminophenoxy)-ethane-N,N,N′,N′-tetraacetic acid, tetraacetoxymethyl ester (BAPTA-AM).

Sequestering agents that contain coordinating oxygen atoms, e.g., O,O-bidentate ligands, generally incorporate those coordinating atoms as hydroxyl (—OH) and/or carbonyl (C═O) moieties. By way of example, for the present purpose, hydroxyl and carbonyl moieties can be covalently protected by the sequestration inactivating agent so that sequestration is temporarily prevented, or can be combined with a sequestration inactivating agent that hydrogen bonds to or otherwise “masks” the ability of the hydroxyl or oxo groups to sequester metal ions. Covalent protection of hydroxyl groups as esters, for instance, may be accomplished using conventional esterification means, such that the sequestration inactivating moiety is the esterifying reagent, the active sequestering agent has free hydroxyl groups, and the inactivated sequestering agent is the esterified form of the sequestering agent. As is understood in the art, sequestering agents containing a diol moiety, e.g., a 1,2-diol or a 1,3-diol, can be protected using suitable diol-protecting reagents as the sequestration inactivating moiety, in which case the active sequestering agent is the unprotected diol, and the inactivated sequestering agent is the protected diol. Carbonyl groups in the metal ion sequestering agent can also be protected and thus inactivated using means known to those of ordinary skill in the art, e.g., by conversion with a sequestration inactivating agent to cyclic acetals or ketals such as 1,3-dioxanes, 1,3-dioxolanes, and the like. Amino groups and other N—H— containing moieties in the sequestering agent can be protected and thus inactivated as amides (e.g., as N-acetylamide, N-benzoylamide, etc.) or by conversion to an alternative N—R group, as is known in the art. See, e.g., Protective Groups in Organic Synthesis, Third Edition, Greene et al., Eds. (Wiley-Interscience, 1999). Cleavage of the association between the metal ion sequestering agent and the sequestration inactivating moiety occurs in vivo as a result of chemical or biochemical reaction with an endogenous molecular entity; for instance, a metal ion sequestering agent inactivated by esterification of hydroxyl groups or by diol protection is activated in vivo as a result of an enzymatic process or a nonenzymatic process, e.g., hydrolysis or, more commonly, via action of hydrogen peroxide.

In a preferred embodiment, the association between the metal ion sequestering agent and the sequestration inactivating moiety involves charge masking, wherein the ability of the coordinating atom or atoms to sequestering metal ions is inactivated by an agent that masks the ionic charge of the coordinating atom or atoms or physically or otherwise prevents a polar coordinating atom in electronically neutral form from sequestering metal ions.

Generally, the metal ion sequestering agents can be divided into two categories, cheators and complexing ligands.

The word chelator comes from the Greek word “chele” which means “claw” or “pincer.” As the name implies, metals that are complexed with chelators form a claw-like structure consisting of one or more molecules. The metal chelate structure may be circular, and may include 5 or 6 member rings that are structurally and chemically stable.

Chelators can be classified by two different methods. One method is by their use: they may be classified as extraction type and color-forming type. Extractions with chelators may be for preparative or analytical purposes. The chelating extraction reaction generally consists of addition of a chelator to a metal-containing solution or material to selectively extract the metal or metals of interest. The color-forming type of chelators—including pyridylazonaphthol (PAN), pyridylazoresorcinol (PAR), thioazoylazoresorcinol (TAR), and many others—have been used in analytical chemistry for many years. The chemistry is similar to that of the extraction type, except that the color-forming chelator will form a distinctive color in the presence or absence of a targeted metal. Generally the types of functional groups that form the chelate complex are similar; however, a color-forming chelator will be water soluble due to the addition of polar or ionic functional groups (such as a sulfonic acid group) to the chelating molecule.

Another method of classifying chelators is according to whether or not the formation of the metal chelate complex results in charge neutralization. Chelators often contain hydronium ions (from a carboxylic acid or hydroxy functional group) that result in charge neutralization, e.g., 8-hydroxyquinoline. Other chelators are non-ionic and simply bind to the metal, thereby conserving the charge of the metal, e.g., ethylene diamine or 1,10-phenanthroline. Chelators sometimes have one acidic group and one basic group which, upon chelation with the metal ion, form a ring structure. Typical acidic groups are carboxylic acid (—COOH), hydroxyl (OH; phenolic or enolic), sulfhydryl (—SH), hydroxylamino (—NH—OH), and arsonic acid (—AsO(OH)₂). Typical basic groups include oxo (═O) and primary, secondary, and tertiary amine groups. Virtually all organic functional groups have been incorporated into chelators.

A complexing ligand may not form a ring structure, but may still be able to form strong complexes with the metal atom. An example of a complexing ligand is cyanide, which can form strong complexes with certain metals such as Fe³⁺ and Cu²⁺. Free cyanide is used to complex and extract gold metal from ore. One or more of the ligands can complex with the metals depending on the ligand and ligand concentration.

It is possible to add selectivity to the complexation reaction. Some metal ion sequestering agents are very selective for a particular metal. For example, dimethylglyoxime forms a planar structure with Ni²⁺ and selectively extracts the metal. Selectivity can be moderated by adjusting the pH. When an acidic group is present, the chelator is made more general by increasing pH and more selective by decreasing the pH. Only metals that form the strongest chelators will form metal chelates under increasingly acidic conditions. As another example, BAPTA selectively chelates calcium ions, EGTA chelates both calcium ions and magnesium ions but is more selective for calcium ions, and EDTA chelates both iron and calcium ions as well as other dicationic and tricationic metal species.

Chelating or ligand complexers may be used in conjunction with other metal chelators to add selectivity. Masking agents are used as an auxiliary complexing agent to prevent the complexation of certain metals so that others can be complexed. Examples of masking agents include sulfosalicylate which masks Al³⁺, cyanide which masks Co²⁺, Ni²⁺, Cu²⁺, Cd²⁺ and Zn²⁺, thiourea which masks Cu²⁺, citrate which masks Al³⁺, Sn⁴⁺ and Zr⁴⁺, and iodide which masks Hg²⁺.

Table 1 indicates some of the more common metal complexers and some of the cations with which they form complexes. In the table, the abbreviations used in the category headings are as follows: E, extraction; CF, color forming; CN, charge neutralizing; and NCN, no charge neutralization.

TABLE 1

| | | | | | Representative ions |
|---|---|---|---|---|---|
| Complexer | E | CF | CN | NCN | complexed |
| 2-Aminoperimidine | x | | | x | SO₄²⁻, Ba²⁺ |
| hydrochloride | | | | | |
| 1-Phenyl-3-methyl-4- | x | | | x | Pu⁴⁺, UO₂²⁺ |
| benzoylpyrazolin-5-one | | | | | |
| Eriochrome black T | | x | x | | Ca²⁺, Mg²⁺, Sr, Zn, Pb |
| Calmagite | | x | x | | Ca²⁺, Mg²⁺, Sr, Zn, Pb |
| o,o-Dihydroxyazobenzene | | x | x | | Ca²⁺, Mg²⁺ |
| Pyridylazonaphthol (PAN) | | x | x | | Bi, Cd, Cu, Pd, Pl, Sn²⁺, UO₂²⁺, |
| | | | | | Hg²⁺, Th, Co, Pb, Fe²⁺, Fe³⁺, Ni²⁺, |
| | | | | | Zn²⁺, La⁺³ |
| Pyridylazonaphthol (PAN) | x | | x | | Alkali metals, Zr⁴⁺, Ge, Ru, Rh, Ir, |
| | | | | | Be, Os |
| Pyridylazo-resorcinol (PAR) | | x | x | | ReO₄⁻, Bi, Cd, Cu, Pd, Pl, Sn²⁺, |
| | | | | | UO₂²⁺, Hg²⁺, Th, Co, Pb, Fe²⁺, Fe^{3+,} |
| | | | | | Ni²⁺, Zn²⁺, La³⁺ |
| Thiazolylazo resorcinol | | x | x | | Pb |
| (TAR) | | | | | |
| 1,10-Phenanthroline | | x | | x | Fe²⁺, Zn, Co, Cu, Cd, SO₄²⁻ |
| 2,2′-Bipyridine | | x | | x | |
| Tripyridine | | x | | x | |
| Bathophenanthroline (4,7- | | | | x | Cu²⁺, Cu⁺, Fe²⁺ |
| diphenyl-1,10-phenanthroline) | | | | | |
| Bathophenanthroline (4,7 | | x | | x | Cu²⁺, Cu⁺, Fe²⁺ |
| diphenyl-2,9-dimethyl-1,10- | | | | | |
| phenanthroline) | | | | | |
| Cuproine | | x | | x | Cu²⁺, Cu⁺, Fe²⁺ |
| Neocuproine | | x | | x | Cu²⁺, Cu⁺, Fe²⁺ |
| 2,4,6-Tripyridyl-S-triazine | | x | | | Fe²⁺ |
| Phenyl-2-pyridyl ketoxime | | x | | | Fe²⁺ |
| Ketoxime | | | x | | |
| Ferrozine | | x | x | | Fe²⁺ |
| Bicinchoninic acid | | | | x | Cu²⁺, Cu⁺ |
| 8-Hydroxyquinoline | x | | x | | Pb, Mg²⁺, Al³⁺, Cu, Zn, Cd |
| 2-Amino-6-sulfo-8- | | x | x | | |
| hydroxyquinoline | | | | | |
| 2-Methyl-8-hydroxyquinoline | x | | x | | Pb, Mg²⁺, Cu, Zn, Cd |
| 5,7-Dichloro 8- | x | | x | | Pb, Mg²⁺, Al³⁺, Cu, Zn, Cd |
| hydroxyquinoline | | | | | |
| Dibromo-8-hydroxyquinoline | x | | x | | Pb, Mg²⁺, Al³⁺, Cu, Zn, Cd |
| Naphthyl azoxine | | x | x | | |
| Xylenol orange | | x | x | | Th⁴⁺, Zr⁴⁺, Bi³⁺, Fe³⁺, Pb²⁺, Zn²⁺, |
| | | | | | Cu²⁺, rare earth metals |
| Calcein (Fluorescein- | | x | x | | Ca²⁺, Mg²⁺ |
| methylene-iminodiacetic acid) | | | | | |
| Pyrocatechol violet | | x | x | | Sn⁴⁺, Zr⁴⁺, Th⁴⁺, UO₂²⁺, Y³⁺, Cd²⁺ |
| Tiron (4,5-Dihydroxy-m- | | x | x | | Al³⁺ |
| benzenedisulfonic acid) | | | | | |
| Alizarin Red S (3,4- | | x | x | | Ca²⁺ |
| dihydroxy-2-anthra- | | | | | |
| quinonesulfonic acid) | | | | | |
| 4-Aminopyridine | x | | | x | |
| Thoron I | | x | | | |
| Arsenazo I | | x | x | | Ca²⁺, Mg²⁺, Th⁴⁺, UO₂²⁺, Pu⁴⁺ |
| Arsenazo III | | x | x | | Ca²⁺, Mg²⁺, Th⁴⁺, UO₂²⁺, Pu⁴⁺, Zr⁴⁺, |
| | | | | | Th⁴⁺ |
| EDTA (ethylenediamine | x | | x | | Fe²⁺, most divalent cations |
| tetraacetic acid) | | | | | |
| CDTA (cyclodiamine | x | | x | | Fe²⁺, most divalent cations |
| tetracetic acid) | | | | | |
| EGTA (ethylene glycol bis (β- | x | | x | | Fe²⁺, most divalent cations |
| aminoethylether)-N,N,N′,N′- | | | | | |
| tetraacetic acid) | | | | | |
| HEDTA (hydroxyethyl- | | | x | | Fe²⁺, most divalent cations |
| ethylenediamine triacetic | | | | | |
| acid) | | | | | |
| DPTA (diethylenetriamine | x | | x | | Fe²⁺, most divalent cations |
| pentaacetic acid) | | | | | |
| DMPS (dimercaptopropane | x | | x | | Fe²⁺, most divalent cations |
| sulfonic acid) | | | | | |
| DMSA (dimercaptosuccinic | x | | x | | Fe²⁺, most divalent cations |
| acid) | | | | | |
| ATPA (aminotrimethylene | x | | x | | Fe²⁺, most divalent cations |
| phosphonic acid) | | | | | |
| CHX-DTPA (Cyclohexyl | x | | x | | Fe²⁺, most divalent cations |
| diethylenetriaminopenta- | | | | | |
| acetate) | | | | | |
| Citric acid | x | | x | | Fe²⁺ |
| 1,2-bis-(2-amino-5- | | x | x | | Ca²⁺, K⁺ |
| fluorophenoxy)ethane- | | | | | |
| N,N,N′,N′-tetraacetic acid | | | | | |
| (5F-BAPTA) | | | | | |
| Desferoxamine | | | | | Fe²⁺ |
| Hydroquinone | x | | x | | Fe²⁺ |
| Benzoquinone | x | | x | | Fe²⁺ |
| dipicrylamine | x | | x | | K⁺ |
| Sodium tetraphenylboron | x | | x | | K⁺ |
| 1,2-dioximes | x | | x | | Ni²⁺, Pd²⁺, Mn²⁺, Fe²⁺, Co²⁺, Ni²⁺, |
| | | | | | Cu²⁺, Zn²⁺ |
| Alpha-furil dioxime | x | | x | | Ni²⁺, Pd²⁺, Mn²⁺, Fe²⁺, Co²⁺, Ni²⁺, |
| | | | | | Cu²⁺, Zn²⁺ |
| Cyclohexanone oxime | x | | x | | Ni²⁺, Pd²⁺, Mn²⁺, Fe²⁺, Co²⁺, Ni²⁺, |
| | | | | | Cu²⁺, Zn²⁺ |
| Cycloheptanone | x | | x | | Ni²⁺, Pd²⁺, Mn²⁺, Fe²⁺, Co²⁺, Ni²⁺, |
| | | | | | Cu²⁺, Zn²⁺ |
| Methyl cyclohexanone- | | x | x | | Ni²⁺, Pd²⁺, Mn²⁺, Fe²⁺, Co²⁺, Ni²⁺, |
| dioxime | | | | | Cu²⁺, Zn²⁺ |
| Ethyl cyclohexanonedioxime | | x | x | | Ni²⁺, Pd²⁺, Mn²⁺, Fe²⁺, Co²⁺, Ni²⁺, |
| | | | | | Cu²⁺, Zn²⁺ |
| Isopropyl 4-cyclohexanone- | | x | x | | Ni²⁺, Pd²⁺, Mn²⁺, Fe²⁺, Co²⁺, Ni²⁺, |
| dioxime | | | | | Cu²⁺, Zn²⁺ |
| Cupferron | x | | x | | M⁴⁺, M⁵⁺, M⁶⁺, Zr⁴⁺, Ga³⁺, Fe³⁺, Ti⁴⁺, |
| | | | | | Hf⁴⁺, U⁴⁺, Sn⁴⁺, Nb⁵⁺, Ta⁵⁺, V⁵⁺, |
| | | | | | Mo⁶⁺, W⁶⁺, Th⁴⁺, Cu²⁺, Bi³⁺ |
| N-Benzolyphenylhydroxyl- | | | x | | Sn⁴⁺, Zi⁴⁺, Ti⁴⁺, Hf⁴⁺, Nb⁵⁺, Ta⁵⁺, |
| amine (BPHA) | | | | | V⁵⁺, Mo⁶⁺, Sb⁵⁺ |
| Arsonic acids | x | | x | | Zr⁴⁺, Ti⁴⁺ |
| Mandelic acid | x | | x | | Zr⁴⁺, Hf⁴⁺ |
| Alpha-nitroso-beta-napthol | x | | x | | Co²⁺, Co³⁺ |
| Anthranilic acid | x | | x | | Ni²⁺, Pb²⁺, Co, Ni²⁺, Cu²⁺, Zn^{2+,} Cd, |
| | | | | | Hg²⁺, Ag⁺ |
| Alpha-benzoinoxime | x | | x | | Cu²⁺ |
| Thionalide | x | | x | | Cu²⁺, Bi³⁺, Hg, As, Sn⁴⁺, Sb⁵⁺, Ag⁺ |
| Tannin | x | | x | | Nb, Ta |
| Ammonium oxalate | x | | x | | Th⁴⁺, Al³⁺, Cr, Fe²⁺, V⁵⁺, Zr⁴⁺, U⁴⁺ |
| Diethyldithio-carbamates | x | | x | | K⁺, most metals |
| 2-Furoic acid | x | | x | | Th⁴⁺ |
| Dimethylglyoxime (DMG) | x | | x | | Ni²⁺, Fe²⁺, Co²⁺, Al³⁺ |
| Isooctylthioglycolic acid | x | | x | | Al³⁺, Fe²⁺, Cu²⁺, Bi³⁺, Sn⁴⁺, Pb²⁺, |
| | | | | | Ag⁺, Hg²⁺ |

The listing of cations in this table should not be taken to be exclusive. Many of these sequestering agents will complex to some extent with many metal cations.

Compounds useful as metal ion sequestering agents herein include any compounds that coordinate to or form complexes with a divalent or polyvalent metal cation, although sequestration of calcium and iron cations is typically preferred for reasons discussed at length earlier herein. Preferred metal ion sequestering agents herein are basic addition salts of a polyacid, e.g., a polycarboxylic acid, a polysulfonic acid, or a polyphosphonic acid, with polycarboxylates particularly preferred.

Suitable metal ion sequestering agents include monomeric polyacids such as EDTA, EGTA, BAPTA, cyclohexanediamine tetraacetic acid (CDTA), hydroxyethyl-ethylenediamine triacetic acid (HEDTA), diethylenetriamine pentaacetic acid (DTPA), dimercaptopropane sulfonic acid (DMPS), dimercaptosuccinic acid (DMSA), aminotrimethylene phosphonic acid (ATPA), citric acid, pharmacologically acceptable salts thereof, and combinations of any of the foregoing. Other exemplary metal ion sequestering agents include: phosphates, e.g., pyrophosphates, tripolyphosphates, and hexametaphosphates; chelating antibiotics such as chloroquine and tetracycline; nitrogen-containing chelating agents containing two or more chelating nitrogen atoms within an imino group or in an aromatic ring (e.g., diimines, 2,2′-bipyridines, etc.); and polyamines such as cyclam (1,4,7,11-tetraazacyclotetradecane), N—(C1-C30 alkyl)-substituted cyclams (e.g., hexadecyclam, tetramethylhexadecylcyclam), diethylenetriamine (DETA), spermine, diethylnorspermine (DENSPM), diethylhomo-spermine (DEHOP), deferoxamine (N′-[5-[[4-[[5-(acetylhydroxyamino)pentyl]amino]-1,4-dioxobutyl]hydroxyamino]pentyl]-N′-(5-aminopentyl)-N-hydroxybutanediamide; also known as desferrioxamine B and DFO), deferiprone, pyridoxal isonicotinoyl hydrazone (PIH), salicylaldehyde isonicotinoyl hydrazone (SIH), ethane-1,2-bis(N-1-amino-3-ethylbutyl-3-thiol).

Additional metal ion sequestering agents which may be useful for the practice of the current disclosure include EDTA-4-aminoquinoline conjugates such as ([2-(bis-ethoxycarbonylmethyl-amino)-ethyl]-{[2-(7-chloro-quinolin-4-ylamino)-ethylcarbamoyl]-methyl}-amino)-acetic acid ethyl ester, ([2-(bis-ethoxycarbonylmethyl-amino)-propyl]-{[2-(7-chloro-quinolin-4-ylamino)-ethylcarbamoyl]-methyl}-amino)-acetic acid ethyl ester, ([3-(bis-ethoxycarbonylmethyl-amino)-propyl]-{[2-(7-chloro-quinolin-4-ylamino)-ethylcarbamoyl]-methyl}-amino)-acetic acid ethyl ester, ([4-(bis-ethoxycarbonylmethyl-amino)-butyl]-{[2-(7-chloro-quinolin-4-ylamino)-ethylcarbamoyl]-methyl}-amino)-acetic acid ethyl ester, ([2-(bis-ethoxymethyl-amino)-ethyl]-{[2-(7-chloro-quinolin-4-ylamino)-ethylcarbamoyl]-methyl}-amino)-acetic acid ethyl ester, ([2-(bis-ethoxymethyl-amino)-propyl]-{[2-(7-chloro-quinolin-4-ylamino)-ethylcarbamoyl]-methyl}-amino)-acetic acid ethyl ester, ([3-(bis-ethoxymethyl-amino)-propyl]-{[2-(7-chloro-quinolin-4-ylamino)-ethylcarbamoyl]-methyl}-amino)-acetic acid ethyl ester, ([4-(bis-ethoxymethyl-amino)-butyl]-{[2-(7-chloro-quinolin-4-ylamino)-ethylcarbamoyl]-methyl}-amino)-acetic acid ethyl ester, as described in Solomon et al. (2006) Med. Chem. 2:133-138.

The metal ion sequestering agent can be included in the compositions herein in amounts ranging from about 0.6 wt. % to about 10 wt. %, for instance, about 1.0 wt. % to about 5.0 wt. %, of the formulation. In certain embodiments, the molar ratio of the sequestration inactivating moiety to the sequestering agent is sufficient to ensure that substantially all sequestering agent molecules are associated with molecules of the sequestration inactivating moiety. Accordingly, in certain embodiments, e.g., when inactivation proceeds via charge masking, the molar ratio of the sequestration inactivating moiety to the sequestering agent is in the range of about 2:1 to about 12:1; for instance, in certain embodiments, the molar ratio of the sequestration inactivating moiety to the sequestering agent may be in the range of about 4:1 to about 10:1; for example, the molar ratio of the sequestration inactivating moiety to the sequestering agent may be in the range of about 6:1 to about 8:1. Specifically, in certain embodiments, the molar ratio of the sequestration inactivating moiety to the sequestering agent is about 8:1.

The disclosure is not, unless otherwise indicated, limited with regard to specific metal ion sequestering agents, and any such agents can be used providing, in general, that they are capable of being buffered to a pH in the range of about 6.5 to about 8.0 and does not interact with any other component of the composition. EDTA and pharmacologically acceptable EDTA salts may be advantageously used. Representative pharmacologically acceptable EDTA salts are typically selected from diammonium EDTA, disodium EDTA, dipotassium EDTA, triammonium EDTA, trisodium EDTA, tripotassium EDTA, and calcium disodium EDTA. EDTA has been widely used as an agent for chelating metals in biological tissue and blood. For example, U.S. Pat. No. 6,348,508 to Denick Jr. et al. describes EDTA as a sequestering agent to bind metal ions. In addition to its use as a chelating agent, EDTA has also been widely used as a preservative in place of benzalkonium chloride, as described, for example, in U.S. Pat. No. 6,211,238 to Castillo et al. U.S. Pat. No. 6,265,444 to Bowman et al. discloses use of EDTA as a preservative and stabilizer.

With respect to the sequestration inactivating moiety, the compound used should be effective to inactivate the sequestering activity of the sequestering agent and preferably facilitate the penetration of the composition components through extra-cellular matrices, tissues, and cell and organelle membranes. An “effective amount” of the sequestration inactivating moiety generally represents a concentration that is sufficient to provide a measurable increase in penetration of one or more of the composition components through extracellular matrices, tissues, and membranes as described herein.

Suitable sequestration inactivating moieties include, by way of example, substances having the formula:

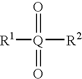

wherein R¹ and R² are independently selected from C₁-C₆ alkyl (preferably C₁-C₃ alkyl), C₁-C₆ heteroalkyl (preferably C₁-C₃ heteroalkyl), C₆-C₁₄ aralkyl (preferably C₆-C₈ aralkyl), and C₂-C₁₂ heteroaralkyl (preferably C₄-C₁₀ heteroaralkyl), and Q is S or P. Compounds wherein Q is S and R¹ and R² are C₁-C₃ alkyl are particularly preferred.

The phrase “having the formula” or “having the structure” is not intended to be limiting and is used in the same way that the term “comprising” is commonly used. With respect to the above structure, the term “alkyl” refers to a linear, branched, or cyclic saturated hydrocarbon group containing 1 to 6 carbon atoms, such as methyl, ethyl, n-propyl, isopropyl, n-butyl, isobutyl, t-butyl, cyclopentyl, cyclohexyl and the like. If not otherwise indicated, the term “alkyl” includes unsubstituted and substituted alkyl, wherein the substituents may be, for example, halo, hydroxyl, sulfhydryl, alkoxy, acyl, etc. The term “alkoxy” intends an alkyl group bound through a single, terminal ether linkage; that is, an “alkoxy” group may be represented as —O-alkyl where alkyl is as defined above. The term “aryl” refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 14 carbon atoms. Exemplary aryl groups are contain one aromatic ring or two fused or linked aromatic rings, e.g., phenyl, naphthyl, biphenyl, diphenylether, diphenylamine, benzophenone, and the like. “Aryl” includes unsubstituted and substituted aryl, wherein the substituents may be as set forth above with respect to optionally substituted “alkyl” groups. The term “aralkyl” refers to an alkyl group with an aryl substituent, wherein “aryl” and “alkyl” are as defined above. Preferred aralkyl groups contain 6 to 14 carbon atoms, and particularly preferred aralkyl groups contain 6 to 8 carbon atoms. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. The term “acyl” refers to substituents having the formula —(CO)-alkyl, —(CO)-aryl, or —(CO)-aralkyl, wherein “alkyl,” “aryl, and “aralkyl” are as defined above. The terms “heteroalkyl” and “heteroaralkyl” are used to refer to heteroatom-containing alkyl and aralkyl groups, respectively, i.e., alkyl and aralkyl groups in which one or more carbon atoms is replaced with an atom other than carbon, e.g., nitrogen, oxygen, sulfur, phosphorus or silicon, typically nitrogen, oxygen or sulfur.

Suitable sequestration inactivating moieties include methylsulfonylmethane (MSM; also referred to as methyl sulfone) and/or combinations of MSM with dimethylsulfoxide (DMSO). MSM is an odorless, highly water-soluble (34% w/v at 79° F.) white crystalline compound with a melting point of 108-110° C. and a molecular weight of 94.1 g/mol. MSM is thought to serve as a multifunctional agent herein, insofar as the agent not only increases the permeability of biological membranes such as cell membranes, but may also facilitate the transport of one or more composition components throughout the layers of the skin (i.e., epidermis, dermis and subcutaneous fat layers), as well as across mucus membranes, endothelial layers, and the like. Furthermore, MSM per se is known to provide medicative effects, and can serve as an anti-inflammatory agent as well as an analgesic. MSM also acts to improve oxidative metabolism in biological tissues, and is a source of organic sulfur, which may assist in the reduction of scarring. MSM additionally possesses beneficial solubilization properties, in that it is soluble in water, as noted above, but exhibits both hydrophilic and hydrophobic properties because of the presence of polar S═O groups and nonpolar methyl groups. The molecular structure of MSM also allows for hydrogen bonding with other molecules, i.e., between the oxygen atom of each S═O group and hydrogen atoms of other molecules, and for formation of van der Waals associations, i.e., between the methyl groups and nonpolar (e.g., hydrocarbyl) segments of other molecules.

The methods and compositions herein may involve use of two or more metal ion sequestering agents used in combination and/or two or more sequestration inactivating agents used in combination. For example, a formulation of the disclosure can contain DMSO in addition to MSM. Since MSM is a metabolite of DMSO (i.e., DMSO is enzymatically converted to MSM), incorporating DMSO into an MSM-containing formulation of the disclosure will tend to gradually increase the fraction of MSM in the formulation. DMSO may also serve as a free radical scavenger, thereby reducing the potential for oxidative damage. If DMSO is added as a secondary enhancer, the amount is preferably in the range of about 1.0 wt. % to 2.0 wt. % of the formulation, and the weight ratio of MSM to DMSO is typically in the range of about 1:1 to about 50:1.

A factor that appears to be related to the performance of the formulations of the disclosure is the molar ratio of the sequestration inactivating moiety to the metal ion sequestering moiety. With charge masking inactivation, for instance using a combination of EDTA and MSM, a molar ratio of at least about 2, for instance, at least about 4, such as at least about 8 may be used. This may be because the formation of further complexes between the sequestration inactivating moiety and the metal ion sequestering agent facilitates the latter's movement to the location of metal cations.

The concentrations of the metal ion sequestering agent and the sequestration inactivating moiety in the present compositions are also of interest. In general, concentrations on the order of a few percent by weight may be used in aqueous vehicles, for example from about 1% to about 8%, such as from about 2% to about 6%. For example, where the sequestration inactivating moiety is MSM and the sequestering agent is EDTA, a concentration of about 2.5 wt % EDTA and about 5 wt % MSM may be used.

It is believed that the sequestration inactivating moiety in formulations of the disclosure may assist in the process of transport of the metal ion sequestering agent, not just into the tissue, but across biological membranes and to the site at which the metal complexer operates. For instance, the sequestration inactivating moiety and metal ion sequestering agent may combine to form a stable moiety that is capable of migrating to a site of operation where the sequestration agent may sequester metal ions, thereby preventing oxidant formation; penetrate protein or lipid aggregates and remove metal ions that provide stability to those aggregates, thereby causing the aggregates to break apart and disperse; and complex intracellular calcium, thereby decreasing the intracellular concentration of free calcium, and consequently, down regulating the caspase and protein kinase C (PKC) pathways.

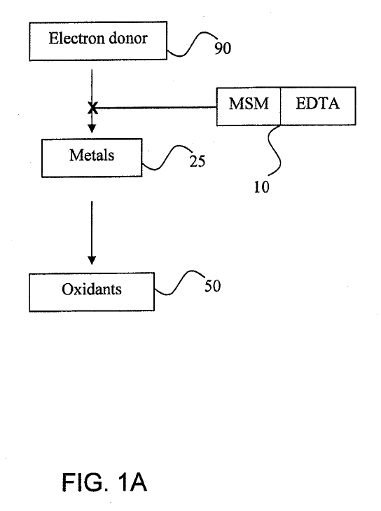

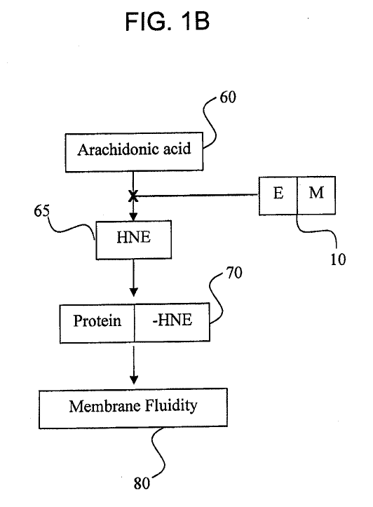

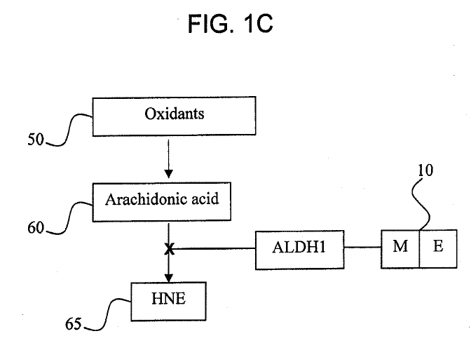

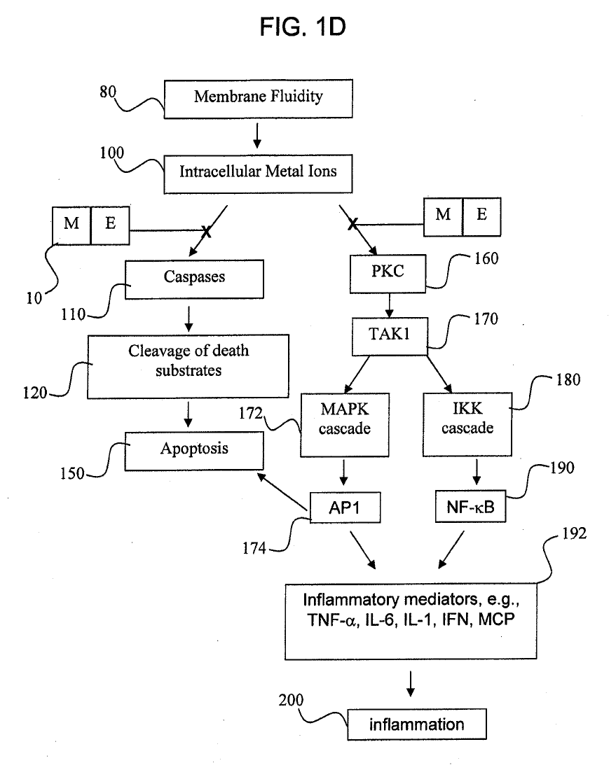

For example, without being bound by theory, and with reference to FIG. 1, a composition 10 of the disclosure including a metal ion sequestering agent, such as EDTA, and a sequestration inactivating moiety, such as MSM, may function in part to prevent and/or at least down regulate or decrease the extra- and intracellular events that otherwise lead to the production of inflammatory mediators, signal cell death, evoke the onset or exacerbation of inflammation, and/or lead to cellular degeneration or unfettered proliferation, and thus, the compositions of the disclosure are useful for the prevention and/or treatment of inflammatory mediated diseases and conditions, such as those described herein.

For instance, with reference to FIG. 1A, a composition 10 of the disclosure including a metal ion sequestering agent, such as EDTA, and a sequestration inactivating moiety, such as MSM, may function in part to sequester extra- or intracellular metal ions 25 that may play an essential role in the production of oxidants 50. For example, various environmental or other such factors or events may lead to the production of electron donors 40 that in the presence of metal ions 25 produce oxidants 50, which oxidants if allowed to propagate may generate a chain reaction that damage cell walls of the surrounding tissues making them more permeable to extracellular metals, such as Ca²⁺.

Specifically, without being bound to theory, by the complexer of the composition complexing metal ions, such as copper, iron, and calcium, which are critical to the pathways for formation and proliferation of free radicals, e.g., in inflamed tissue, the metal ion sequestering agent preferentially binds to metal ions so as to form complexes therewith that are flushed into the bloodstream and excreted. In this way, the production of oxygen free radicals, reactive oxygen species (ROS), and reactive molecular fragments is reduced, in turn reducing pathological lipid peroxidation of cell membranes, and/or damage to DNA, structural proteins, lipoproteins, lipids, and/or enzymes typically caused by ROS and the like.

For instance, with reference to FIG. 1B, under oxidative stress, oxidants 50, such as free radicals, initiate peroxidation of membrane lipids, e.g. arachidonic acid 60 (PUFA). This process may form highly reactive and toxic lipid aldehydes (LDAs). A major product of such a reaction is the formation of 4-hydroxynonenal 65 (HNE), which is highly reactive and cytotoxic at micromolar concentrations. HNE is particularly deleterious to membrane proteins and has been associated with apoptosis of epithelial cells. For example, HNE 65 may interact with various lipids and/or proteins within the cell membrane to produce protein-HNE adducts 70, the formation of which leads to increased membrane fluidity 80.

An MSM and EDTA composition of the disclosure may prevent this by sequestering metal ions such as Fe²⁺ or Fe³⁺, which are essential for the conversion of arachidonic acid to 4-HNE. Specifically, a composition of the disclosure may function at least in part to sequester metal ions such as Fe²⁺ and thereby disrupt the pathway for the conversion of arachidonic acid in cellular membranes to 11-hydroperoxide (and the various free-radical analogs thereof) and 11-hydroperoxide to 4-hydroxynonenal.

Accordingly, during instances of oxidative stress and/or inflammation, reactive oxygen species may be produced which can damage various lipids and/or proteins of cell membranes of the body, which damaged lipids and/or proteins may form lipid/protein deposits, in turn forming aggregates bound by metal ions, such as calcium. A composition of the disclosure, including a metal ion sequestering agent, such as a chelating agent disclosed herein, is capable of binding the metal ions forming lipid aggregates, thereby chelating the metal ion, dissolving the lipid deposits, and allowing the freed lipids to be cleared, for example, by the liver, thereby protecting the integrity of the cell membrane.

Additionally, as can be seen with reference to FIG. 1C, a composition of the disclosure 10 may function in part to directly or indirectly activate the production of aldehyde dehyrdogenase 1 90 (ALDH1). Specifically, a composition of the disclosure may function at least in part to increase or upregulate the intracellular transcription and production of ALDH1 90, which ALDH1 functions to oxidize 4-HNE 65 to HNA 68, for instance, in the presence of nicotinamide adenine dinucleotide (NAD), thereby detoxifying 4-HNE and preventing the damaging effects of 4-HNE, such as its role in the production of Protein-HNE conjugates 60 that lead to increased membrane fluidity 80. Thus a composition of the disclosure is effective for inhibiting protein-HNE formation and protecting membrane integrity.

As can be seen with reference to FIG. 1D, increased membrane fluidity 80, may lead to the cell membrane becoming more permeable to extracellular metal ions, such as Ca²⁺, which results in an increase in the intracellular concentration of such metal ions. For example, an increase in Ca²⁺ levels 100, may lead to the activation of the caspase pathway 110, which may result in cellular apoptosis 150, and/or may lead to the activation of the protein kinase C 160 (PKC) pathway, leading to the production and release of pro-inflammatory mediators 200, such as TNF-α, IL-1, IL-6, MMPs, MCP, IFN, and the like, which may lead to the onset of inflammation that if left untreated may result in one or more of the inflammatory diseases set forth herein.

Accordingly, a composition of the disclosure 10 may function to sequester intracellular metal ions 100, such as Ca²⁺, thereby preventing such metal ions 100 from activating one or more members of the various caspase family 110, which caspases may function to cleave the death substrates 120, e.g., lamin and/or poly (ADP-ribose) polymerase (PARP), which substrates may otherwise signal for programmed cell death, thereby leading to apoptosis 150 of the cell. Thus a composition of the disclosure is effective for reducing caspase (e.g., caspase-3 concentrations), and thereby preventing cell death.

Further, a composition of the disclosure 10 may function to sequester intracellular metal ions 100, such as Ca²⁺, thereby preventing such metal ions 100 from activating PKC 160. Typically, PKC 160 when activated, may function to activate the TAK1 pathway 170, which pathway may lead to the activation of the mitogen-activated protein kinase (MAPK) cascade 172 and/or the activation of the IKK cascade 180, one or more of the components thereof may signal for programmed cell death, thereby leading to apoptosis 150 of the cell, and/or the release of inflammatory mediators 192, such as TNF-α, IL-1, IL-6, MMPs, MCP, IFN, and the like, which in turn may lead to the onset of inflammation 200 that if left untreated may result in one or more of the inflammatory diseases set forth herein.

For instance, by preventing the activation of the TAK1 170 pathway, a composition 10 of the disclosure may prevent the activation of mitogen-activated protein kinase (MAPK) cascade 172 thereby preventing the activation of C-Jun N-terminal kinases (JNK) and P38 mitogen-activated protein kinases (P38), resulting in a down regulation of the transcription and production of AP1 174, which AP1 may otherwise act as a signal for apoptosis 150 or inflammation 200. Additionally, by preventing the activation of the TAK1 pathway 170, a composition 10 of the disclosure may prevent the activation of the IKK cascade 180, which prevents the activation of NIK and IKK, which in turn can result in a down regulation of the transcription and production of Nf-κB, which Nf-κB may otherwise act as a signal for apoptosis 150 or inflammation 200.

Additionally, without being bound by theory, an additional role played by the metal ion sequestering agent is in the removal of active sites of metalloproteinases (MMPs) in the tissue, such as inflamed tissue, by sequestration of the enzymes' metal center. By inactivating metalloproteinases in this way, the sequestration agent may slow or stop the degeneration of protein complexes within the inflamed tissue, thereby providing an opportunity for the tissues to rebuild themselves.

Accordingly, a composition of the disclosure, including a metal ion sequestering agent and a sequestration inactivating agent, is multifunctional in the context of the present disclosure, insofar as the formulation serves to decrease unwanted proteinase (e.g., collagenase) activity, prevent formation of lipid and/or protein deposits, and/or reduces lipid and/or protein deposits that have already formed, prevent oxidative stress, quench cell death and/or inflammatory cascades, thereby preventing and/or treating the deleterious effects thereof.

The formulations herein may consist essentially of the metal ion sequestering agent and the sequestration inactivating moiety, such that no additional therapeutic agents are incorporated, although various excipients, carriers, preservatives, and the like will typically be present.

In an alternative embodiment, the composition may include an added anti-inflammatory agent in a therapeutically or prophylactically effective amount (as explained elsewhere herein, the term “therapeutic” is generally intended to encompass “prophylactic” use as well).

Any suitable anti-inflammatory agent in any suitable amount may be used so long as the anti-inflammatory agent is capable of being combined with the metal ion sequestering agent and/or sequestration inactivating moiety components to form a composition that is capable of preventing and/or treating inflammation and/or an inflammatory related pathology. Thus, in certain embodiments, the present disclosure includes a composition comprising one or more metal ion sequestering agents, one or more sequestration inactivating moieties, and one or more anti-inflammatory compounds. Accordingly, a suitable anti-inflammatory agent may be one or more of those described herein below.

Non-steroid anti-inflammatory drugs are suitable compounds for use in the instant disclosure and include, naproxen (such as Aleve, Naprosyn), sulindac (such as Clinoril), tolmetin (such as Tolectin), ketorolac (such as Toradol), celecoxib (such as Celebrex), ibuprofen (such as Advil, Motrin, Medipren, Nuprin), diclofenac (such as Voltaren, Cataflam, Voltaren-XR), acetylsalicylic acid, nabumetone (such as Relafen), etodolac (such as Lodine), indomethacin (such as Indocin, Indocin-SR), piroxicam (such as Feldene), cox-2 Inhibitors, ketoprofen (Orudis, Oruvail), antiplatelet medications, salsalate (such as Disalcid, Salflex), valdecoxib (such as Bextra), oxaprozin (Daypro), diflunisal (such as Dolobid), meclofenamate (such as Meclomen) and flurbiprofen (such as Ansaid). It is understood that derivatives of the above, such as salts, polymorphs and the like are suitable for use in the composition.

Other suitable bioactive agents including anti-inflammatory agents based on the use of corticosteroids and leukotrienes are suitable. These include, but are not limited to, oral (and intravenous) corticosteroids (systemic corticosteroids), inhaled corticosteroids, and leukotriene modifiers (Accolate and Singular).

Suitable examples of oral or intravenous corticosteroids include, but are not limited to cortisone, hydrocortisone (such as Cortef), prednisone (such as Deltasone, Meticorten, Orasone), prednisolone (such as Delta-Cortef, Pediapred, Prelone), triamcinolone (such as Aristocort, Kenacort), methylprednisolone (such as Medrol, Methylpred, Solu-Medrol), dexamethasone (such as Decadron, Dexone, Hexadrol), betamethasone (such as Celestone) and the like. Suitable inhaled corticosteroids include but are not limited to beclomethasone (such as Beclovent, Beconase, Vanceril, Vancenase), budesonide (such as Pulmicort, Rhinocort), mometasone (such as Nasonex), triamcinolone (such as Azmacort, Nasacort), flunisolide (such as AeroBid, Nasalide, Nasarel), and fluticasone (such as Flovent, Flonase).

Other suitable anti-inflammatory agents include some combination medications that include a corticosteroid plus a long acting bronchodilator drug (e.g., Advair), mineralocorticoids, carboxyamidotriazole, combretastatin A-4, squalamine, 6-O-chloroacetyl-carbonyl)-fumagillol, thalidomide, angiostatin, troponin-1, angiotensin II antagonists, hydroxychloroquinone, penicillamine, sulfasalazine, leukotriene modifiers such as but not limited to Accolate, Singulair, Zyflo and the like.

More specifically, the anti-inflammatory compound can be selected from the group consisting of the following:

(a) Leukotriene biosynthesis inhibitors, 5-lipoxygenase (5-LO) inhibitors, and 5-lipoxygenase activating protein (FLAP) antagonists, including, zileuton; ABT-761; fenleuton; tepoxalin; Abbott-79175; Abbott-85761; N-(5-substituted)-thiophene-2-alkylsulfonamides; 2,6-di-tert-butylphenol hydrazones; Zeneca ZD-2138; SB-210661; pyridinyl-substituted 2-cyanonaphthalene compound L-739,010; 2-cyanoquinoline compound L-746,530; indole and quinoline compounds MK-591, MK-886, and BAY x 1005;

(b) Receptor antagonists for leukotrienes LTB4, LTC4, LTD4, and LTE4, including phenothiazin-3-one compound L-651,392; amidino compound CGS-25019c; benzoxazolamine compound ontazolast; benzenecarboximidamide compound BIIL 284/260; compounds zafirlukast, ablukast, montelukast, pranlukast, verlukast (MK-679), RG-12525, Ro-245913, iralukast (CGP 45715A), and BAY x 7195;

(c) 5-Lipoxygenase (5-LO) inhibitors; and 5-lipoxygenase activating protein (FLAP) antagonists;

(d) Dual inhibitors of 5-lipoxygenase (5-LO) and antagonists of platelet activating factor (PAF);

(e) Leukotriene antagonists (LTRAs) of LTB4, LTC4, LTD4, and LTE4;

(f) Antihistaminic H1 receptor antagonists, including, cetirizine, loratadine, desloratadine, fexofenadine, astemizole, azelastine, and chlorpheniramine;

(g) Gastroprotective H2 receptor antagonists;

(h) α₁- and α₂-adrenoceptor agonist vasoconstrictor sympathomimetic agents administered orally or topically for decongestant use, including propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride;

(i) one or more α₁- and α₂-adrenoceptor agonists as recited in (h) above in combination with one or more inhibitors of 5-lipoxygenase (5-LO) as recited in (a) above;

(j) Theophylline and aminophylline;

(k) Sodium cromoglycate;

(l) Muscarinic receptor (M1, M2, and M3) antagonists;

(m) COX-1 inhibitors (NTHEs); and nitric oxide NTHEs;

(n) COX-2 selective inhibitor for example rofecoxib and celecoxib;

(o) COX-3 inhibitor for example acetaminophen;

(p) insulin-like growth factor type I (IGF-1) mimetics;

(q) Ciclesonide;

(r) Corticosteroids, including prednisone, methylprednisone, triamcinolone, beclomethasone, fluticasone, budesonide, hydrocortisone, dexamethasone, mometasone furoate, azmacort, betamethasone, beclovent, prelone, prednisolone, flunisolide, trimcinolone acetonide, beclomethasone dipropionate, fluticasone propionate, mometasone furoate, solumedrol and salmeterol;

(s) Tryptase inhibitors;

(t) Platelet activating factor (PAF) antagonists;

(u) Monoclonal antibodies active against endogenous inflammatory entities;

(v) IPL 576;

(w) Anti-tumor necrosis factor (TNF-α) agents, including etanercept, infliximab, and D2E7;

(x) DMARDs for example leflunomide;

(y) Elastase inhibitors, including UT-77 and ZD-0892;

(z) TCR peptides;

(aa) Interleukin converting enzyme (ICE) inhibitors;

(bb) IMPDH inhibitors;

(cc) Adhesion molecule inhibitors including VLA-4 antagonists;

(dd) Cathepsins;

(ee) Mitogen activated protein kinase (MAPK) inhibitors;

(ff) Mitogen activated protein kinase kinase (MAPKK) inhibitors;

(gg) Glucose-6 phosphate dehydrogenase inhibitors;

(hh) Kinin-B1- and B2-receptor antagonists;

(ii) Gold in the form of an aurothio group in combination with hydrophilic groups;

(jj) Immunosuppressive agents, including cyclosporine, azathioprine, tacrolimus, and methotrexate;

(kk) Anti-gout agents, including colchicine;

(ll) Xanthine oxidase inhibitors, including allopurinol;

(mm) Uricosuric agents, including probenecid, sulfinpyrazone, and benzbromarone;

(nn) Antineoplastic agents that are antimitotic drugs for example vinblastine, vincristine, cyclophosphamide, and hydroxyurea;

(oo) Growth hormone secretagogues;

(pp) Inhibitors of matrix metalloproteinases (MMPs), including the stromelysins, the collagenases, the gelatinases, aggrecanase, collagenase-1 (MMP-1), collagenase-2 (MMP-8), collagenase-3 (MMP-13), stromelysin-1 (MMP-3), stromelysin-2 (MMP-10), and stromelysin-3 (MMP-11);

(qq) Transforming growth factor (TGF-β);

(rr) Platelet-derived growth factor (PDGF);

(ss) Fibroblast growth factor, including basic fibroblast growth factor (bFGF);

(tt) Granulocyte macrophage colony stimulating factor (GM-CSF);

(uu) Capsaicin;

(vv) Tachykinin NK1 and NK3 receptor antagonists, including NKP-608C; SB-233412 (talnetant); and D-4418; and

(ww) A2A receptor agonist, or any combinations thereof.

In addition to medical drugs, including but not limited to those described above, many herbs have anti-inflammatory qualities, including hyssop, ginger, Arnica montana which contains helenalin, a sesquiterpene lactone, and willow bark, which contains salicylic acid, a substance related to the active ingredient in aspirin. These herbs are encompassed by the present disclosure and one or more herbs can be combined in a composition with one or more chelators and one or more sequestration inactivating moieties.

The chelator, sequestration inactivating moiety, and/or anti-inflammatory compound may be administered either simultaneously or one after another in any order so as to be effective in treating or preventing any inflammatory condition, disorder or disease. In certain embodiments, one or more antioxidants may be included in a composition of the present disclosure, such as NAC, ascorbic acid, vitamin E, and the like.

A variety of means can be used to formulate the compositions of the present disclosure. Techniques for pharmaceutical formulation and administration may be found in “Remington: The Science and Practice of Pharmacy,” Twentieth Edition, Lippincott Williams & Wilkins, Philadelphia, Pa. (1995). For human or animal administration, preparations should meet sterility, pyrogenicity, and general safety and purity standards comparable to those required by the FDA. Administration of the pharmaceutical composition can be performed in a variety of ways, as described herein.

The amount of the composition administered and the relative amounts of each component therein (e.g., metal ion sequestering agent, sequestration inactivating moiety, anti-inflammatory agent, etc.) will depend on a number of factors and will vary from subject to subject and depend on, for example, the particular disorder or condition being treated, the severity of the symptoms, the subject's age, weight and general condition, and the judgment of the prescribing physician.

The term “dosage form” denotes any form of a pharmaceutical composition that contains an amount of active agent sufficient to achieve a therapeutic effect with a single administration. When the composition is a tablet or capsule, the dosage form is usually one such tablet or capsule. The frequency of administration that will provide the most effective results in an efficient manner without overdosing will vary with the characteristics of the particular active agent, including both its pharmacological characteristics and its physical characteristics, such as hydrophilicity.

The compositions of the present disclosure can also be formulated for controlled release or sustained release. The term “controlled release” refers to a drug-containing formulation or fraction thereof in which release of the drug is not immediate, e.g., with a “controlled release” formulation, administration does not result in immediate release of the drug into an absorption pool. The term is used interchangeably with “nonimmediate release” as defined in Remington: The Science and Practice of Pharmacy, cited previously. In general, the term “controlled release” as used herein includes sustained release and delayed release formulations. The term “sustained release” (synonymous with “extended release”) is used in its conventional sense to refer to a drug formulation that provides for gradual release of a drug over an extended period of time, and that preferably, although not necessarily, results in substantially constant blood levels of a drug over an extended time period.

The present formulations may also include conventional additives such as opacifiers, antioxidants, fragrance, colorant, gelling agents, thickening agents, stabilizers, surfactants, and the like. Other agents may also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof.

Administration of a compound of the disclosure may be carried out using any appropriate mode of administration. Thus, administration can be, for example, oral, parenteral, topical, transdermal, transmucosal (including rectal and vaginal), sublingual, by inhalation, or via an implanted reservoir in a dosage form.

Depending on the intended mode of administration, the pharmaceutical formulation may be a solid, semi-solid or liquid, such as, for example, a tablet, a capsule, a caplet, a liquid, a suspension, an emulsion, a suppository, granules, pellets, beads, a powder, or the like, preferably in unit dosage form suitable for single administration of a precise dosage. Suitable pharmaceutical compositions and dosage forms may be prepared using conventional methods known to those in the field of pharmaceutical formulation and described in the pertinent texts and literature, e.g., in Remington: The Science and Practice of Pharmacy, supra.

The dosage regimen will depend on a number of factors that may readily be determined, such as severity of the condition and responsiveness of the condition to be treated, but will normally involve one or more doses per day, with a course of treatment lasting from several days to several months, or until a cure is effected or a diminution of disease state is achieved. One of ordinary skill may readily determine optimum dosages, dosing methodologies, and repetition rates. Specific formulations directed to specified routes of administration are described herein.

For orally active formulations of the disclosure, oral administration is preferred.

Oral dosage forms, as is well known in the art, include tablets, capsules, caplets, solutions, suspensions and syrups, and may also comprise a plurality of granules, beads, powders, or pellets that may or may not be encapsulated. Such compositions and preparations should contain at least 0.1% of the inactivated metal ion sequestering agent, typically in the range of about 2 wt. % to about 75 wt. %, and most usually in the range of about 25 wt. % to about 60 wt. %. Preferred oral dosage forms are tablets and capsules.

Tablets may be manufactured using standard tablet processing procedures and equipment. Direct compression and granulation techniques are preferred. In addition to the active agent, tablets will generally contain inactive, pharmaceutically acceptable carrier materials such as binders, lubricants, disintegrants, fillers, stabilizers, surfactants, coloring agents, and the like. Binders are used to impart cohesive qualities to a tablet, and thus ensure that the tablet remains intact. Suitable binder materials include, but are not limited to, starch (including corn starch and pregelatinized starch), gelatin, sugars (including sucrose, glucose, dextrose, and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Lubricants are used to facilitate tablet manufacture, promoting powder flow and preventing particle capping (i.e., particle breakage) when pressure is relieved. Useful lubricants are magnesium stearate, calcium stearate, and stearic acid. Disintegrants are used to facilitate disintegration of the tablet, and are generally starches, clays, celluloses, algins, gums, or crosslinked polymers. Fillers include, for example, materials such as silicon dioxide, titanium dioxide, alumina, talc, kaolin, powdered cellulose, and microcrystalline cellulose, as well as soluble materials such as mannitol, urea, sucrose, lactose, dextrose, sodium chloride, and sorbitol. Stabilizers, as well known in the art, are used to inhibit or retard drug decomposition reactions that include, by way of example, oxidative reactions.

Capsules may also be used as an oral dosage form for those compounds that are orally active, in which case the active agent-containing composition may be encapsulated in the form of a liquid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington: The Science and Practice of Pharmacy, cited supra, which describes materials and methods for preparing encapsulated pharmaceuticals.

Oral dosage forms, whether tablets, capsules, caplets, or particulates, may, if desired, be formulated so as to provide for gradual, sustained release of the active agent over an extended time period. Generally, as will be appreciated by those of ordinary skill in the art, sustained release dosage forms are formulated by dispersing the active agent within a matrix of a gradually hydrolyzable material such as a hydrophilic polymer, or by coating a solid, drug-containing dosage form with such a material. Hydrophilic polymers useful for providing a sustained release coating or matrix include, by way of example: cellulosic polymers such as hydroxypropyl cellulose, hydroxyethyl cellulose, hydroxypropyl methyl cellulose, methyl cellulose, ethyl cellulose, cellulose acetate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate; and vinyl polymers and copolymers such as polyvinyl pyrrolidone, polyvinyl acetate, and ethylene-vinyl acetate copolymer.

When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A sweetening agent, such as sucrose, lactose or saccharin may be added or a flavoring agent, such as peppermint, oil of wintergreen, or cherry flavoring, may be present. A syrup or elixir may contain the active compound, sucrose as a sweetening agent, methyl and propylparabensas preservatives, a dye and flavoring, such as cherry or orange flavor.

The compositions of the present disclosure can also be administered parenterally to a subject/patient in need of such treatment. The term “parenteral” generally encompasses any mode of administration other than oral administration, but typically, and as used herein, refers primarily to subcutaneous, intravenous, and intramuscular injection.

Preparations according to this disclosure for parenteral administration include sterile aqueous and nonaqueous solutions, suspensions, and emulsions. Injectable aqueous solutions contain the active agent in water-soluble form. Examples of nonaqueous solvents or vehicles include fatty oils, such as olive oil and corn oil, synthetic fatty acid esters, such as ethyl oleate or triglycerides, low molecular weight alcohols such as propylene glycol, synthetic hydrophilic polymers such as polyethylene glycol, liposomes, and the like. Parenteral formulations may also contain adjuvants such as solubilizers, preservatives, wetting agents, emulsifiers, dispersants, and stabilizers, and aqueous suspensions may contain substances that increase the viscosity of the suspension, such as sodium carboxymethyl cellulose, sorbitol, and dextran. Injectable formulations are rendered sterile by incorporation of a sterilizing agent, filtration through a bacteria-retaining filter, irradiation, or heat. They can also be manufactured using a sterile injectable medium. The active agent may also be in dried, e.g., lyophilized, form that may be rehydrated with a suitable vehicle immediately prior to administration via injection.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredients plus any additional desired ingredients from a previously sterile-filtered solution thereof.

The preparation of more, or highly, concentrated solutions for subcutaneous or intramuscular injection is also contemplated. In this regard, the use of DMSO as solvent is preferred as this will result in extremely rapid penetration, delivering high concentrations of the active compound(s) or agent(s) to a small area.

The compositions of the present disclosure can be administered topically to a subject/patient in need of such treatment. The term “topical administration” is used in its conventional sense to mean delivery (e.g., process of applying or spreading one or more compositions according to the instant disclosure onto the surface of the skin) to a predetermined area of skin or mucosa of a subject in need thereof, as in, for example, the treatment of various skin disorders. Topical administration, in contrast to transdermal administration, is intended to provide a local rather than a systemic effect. In certain instances, as may be stated or implied by the circumstances, the terms “topical drug administration” and “transdermal drug administration” may be used interchangeably.

By “predetermined area” of skin or mucosal tissue, which refers to the area of skin or mucosal tissue through which a drug-enhancer formulation is delivered, is intended a defined area of intact unbroken living skin or mucosal tissue, or in certain instances, broken skin, such as skin that includes an abrasion or cut. That area will usually be in the range of about 5 cm² to about 200 cm², more usually in the range of about 5 cm² to about 100 cm², preferably in the range of about 20 cm2 to about 60 cm2. However, it will be appreciated by those skilled in the art of drug delivery that the area of skin or mucosal tissue through which drug is administered may vary significantly, depending on patch configuration, dose, and the like.

Suitable formulations for topical administration include ointments, creams, gels, lotions, pastes, and the like.

Formulations may also be prepared with liposomes, micelles, and microspheres.

Topical formulations may also contain irritation-mitigating additives to minimize or eliminate the possibility of skin irritation or skin damage resulting from the pharmacologically active base or other components of the composition. Suitable irritation-mitigating additives include, for example: α-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. The irritant-mitigating additive, if present, may be incorporated into the present compositions at a concentration effective to mitigate irritation or skin damage, typically representing not more than about 20 wt. %, more typically not more than about 5 wt. %, of the composition.

The pharmaceutical compositions of the present disclosure can be administered to a subject/patient in need of such prevention or treatment using a transdermal delivery system, e.g., a topical or transdermal “patch.” By “transdermal” delivery may be meant administration of a formulation to the skin surface of an individual so that the formulation passes through the skin tissue and into the individual's blood stream, thereby providing a systemic effect. The term “transdermal” is intended to include “transmucosal” drug administration, e.g., administration of a drug to the mucosal (e.g., sublingual, buccal, vaginal, rectal) surface of an individual so that the drug passes through the mucosal tissue and into the individual's blood stream. Transdermal, dependent on the context, may also include nasal delivery, such as, administration through the nose and/or the mucosa thereof.

The transdermal patch contains the active agent within a laminated structure that is to be affixed to the skin. In such a structure, the pharmaceutical composition is contained in a layer, or “reservoir,” underlying an upper backing layer. The laminated structure may contain a single reservoir, or it may contain multiple reservoirs.

The reservoir can comprise a polymeric matrix of a pharmaceutically acceptable adhesive material that serves to affix the system to the skin during drug delivery; typically, the adhesive material is a pressure-sensitive adhesive (PSA) that is suitable for long-term skin contact, and which should be physically and chemically compatible with the pharmaceutical composition and any carriers, vehicles or other additives that are present. Examples of suitable adhesive materials include, but are not limited to, the following: polyethylenes; polysiloxanes; polyisobutylenes; polyacrylates; polyacrylamides; polyurethanes; plasticized ethylene-vinyl acetate copolymers; and tacky rubbers such as polyisobutene, polybutadiene, polystyrene-isoprene copolymers, polystyrene-butadiene copolymers, and neoprene (polychloroprene).

The compositions of the present disclosure can also be administered nasally to a subject/patient in need of such treatment. The term “nasal” as used herein is intended to encompass delivery through the mucosa of the nasal cavity, throat, and/or lungs. For instance, formulations for nasal administration can be prepared with standard excipients, e.g., as a solution in saline, as a dry powder, or as an aerosol and may be administered by a metered dose inhaler (MDI), dry powder inhaler (DPI) or a nebulizer.

For example, a composition of the present disclosure may be formulated for inhalation and therefore be adapted to be administered via an inhaler. For instance, the composition may be formulated in solution and maintained in a pressurized canister with a hand operated actuator, such as a suitable inhaler. A suitable inhaler may be, for example, a metered-dose inhaler (MDI) whereupon activation a fixed dose of the present composition is released in aerosol form.

In addition to the compositions described previously, the composition of the disclosure may also be formulated as a depot preparation for controlled release of the active agent, preferably sustained release over an extended time period. These sustained release dosage forms are generally administered by implantation (e.g., subcutaneously or by intramuscular injection). Although the present compositions will generally be administered orally, parenterally, topically, transdermally, or via an implanted depot, other modes of administration are suitable as well. For example, administration may be rectal or vaginal, preferably using a suppository that contains, in addition to the active agent, excipients such as a suppository wax. For suppositories, traditional binders and carriers may include, for example, polyalkylene glycols or triglycerides; such suppositories may be formed from mixtures containing the active ingredient in the range of 0.5% to 10%, preferably 1%-2%.

### EXAMPLES

The following examples are put forth so as to provide those skilled in the art with a complete disclosure and description of how to make and use embodiments in accordance with the disclosure, and are not intended to limit the scope of what the inventors regard as their discovery. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example I

In accordance with the methods of the disclosure, Lewis or Sprague Dawley rats were used to examine the effects of acute inflammation. In this experiment, Lipopolysaccharide (LPS) was used as a prototypical endotoxin for the promotion of pro-inflammatory cytokine secretion by exposing the rats to LPS so as to induce an LPS challenge therein.

Specifically, 10 mg/kg body weight of LPS in saline was injected intravenously into the rats via the tail vein. Controls were injected with composition of saline only. A twenty μl composition of MSM and EDTA (e.g., 5.4% MSM+2.6% EDTA) was then administered to the rats via the nasal route 15 minutes after the LPS-injection and then after every two hrs. After six hours, the rats were sacrificed and assessed for inflammation.

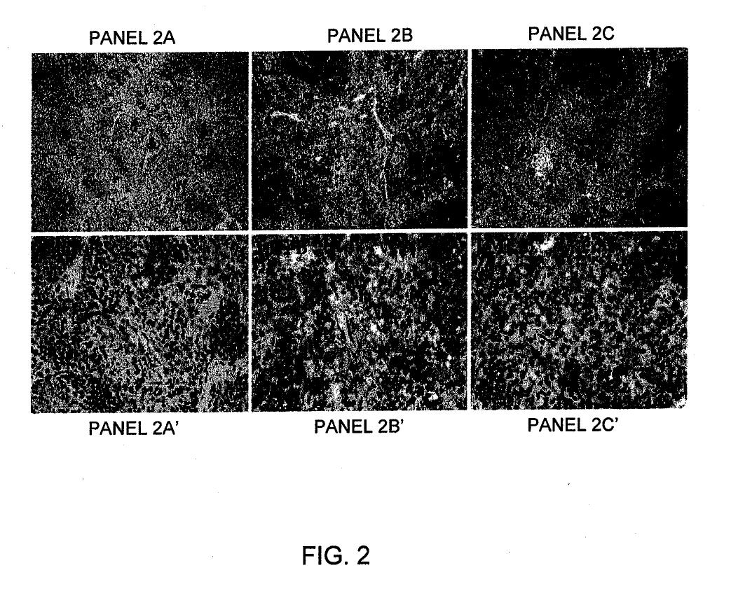

FIG. 2 shows rat spleen after 6 hours of saline only treatment, saline+LPS treatment, and MSM+EDTA treatment. Accordingly, the various panels represent immunohistochemical analysis of paraffin-embedded rat spleen. Panels 2A and 2A′ represent control spleen samples. Panels 2B and 2B′ represent rat spleen samples after injection with LPS and the onset of LPS challenge, but before treatment with a MSM and EDTA composition. Panels 2C and 2C′ represent rat spleen samples post injection of LPS and after the administration of an MSM and EDTA composition. TNF-α is shown as dark dot signal. As can be seen with reference to Panels 2B and 2B′, the spleen showed increased (intense) signal of the inflammatory cytokine, TNF-α, in the LPS-treated rats. As can be seen with reference to Panels 2C and 2C′, this inflammation was ameliorated by the administration of the MSM and EDTA composition. Consequently, as can be seen with reference to FIG. 2, the intense immunoreactivity observed in the LPS-injected group was significantly reduced in the LPS and MSM+EDTA treated group.

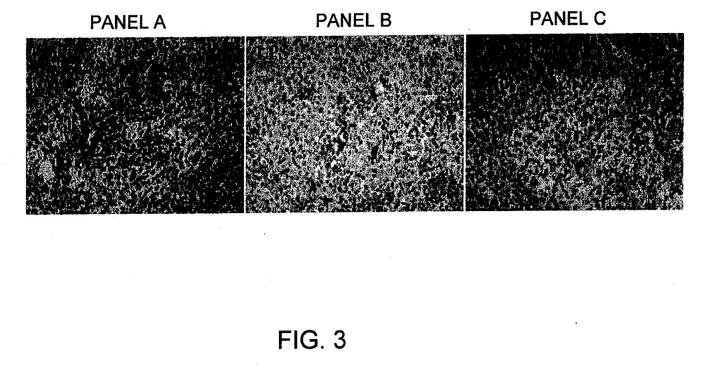

FIG. 3 shows rat spleen after 6 hours of saline only treatment, saline+LPS treatment, and MSM+EDTA treatment. Accordingly, the various panels represent immunohistochemical analysis of paraffin-embedded rat spleen. Panel 3A represents control spleen sample. Panel 3B represents rat spleen sample after injection with LPS and the onset of LPS challenge, but before treatment with a MSM and EDTA composition. Panel 3C represents rat spleen sample post injection of LPS and after the administration of an MSM and EDTA composition.

The dark dot signal in the samples shows cytoplasmic and perinuclear localization of caspase-3 in apoptotic cells. As can be seen with reference to Panel 3A some endogenous apoptosis can be seen in the normal spleen. As can be seen with reference to Panel 3B, significant apoptosis can be observed in the LPS injected group. As can be seen with reference to Panel 3C, apoptosis was significantly reduced in the LPS and MSM+EDTA treated group.

### Example II

In accordance with the methods of the disclosure, Lewis or Sprague Dawley rats were used to examine the effects of chronic inflammation. In this experiment, a streptozotocin-induced rat model was used to assess inflammatory conditions with a group of diabetic rats being a model for the effects of inflammation. Both normal (NR) and diabetic (DR) rats were dosed orally with an MSM and EDTA composition. The concentration of the MSM was 0.0054% (approximately 560 μM) and the concentration of EDTA was 0.0026% (approximately 70 μM). The rats were sacrificed after 45 days.

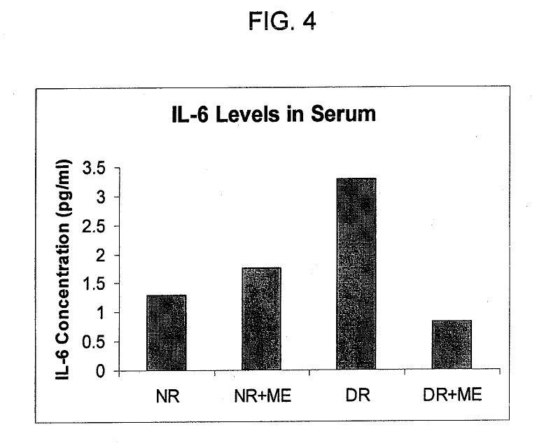

FIG. 4 presents a bar graph illustrating serum IL-6 levels. As can be seen with reference to FIG. 4, the inflammatory cytokine IL-6 was increased in the diabetic rat (DR), while in the MSM and EDTA treated rat this increase was ameliorated.

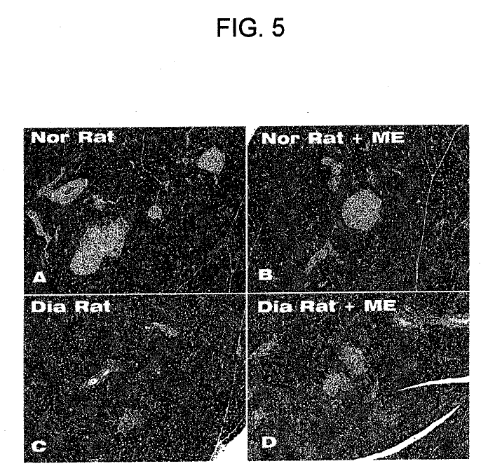

FIG. 5 presents a low magnification (100×) photomicrograph of a pancreatic lobule. FIG. 5 shows a 4 μm section of formalin-fixed, paraffin-embedded pancreas that is H&E stained. As can be seen with reference to Panel A, a section of the pancreas from normal rat dosed orally with water without an MSM and EDTA composition shows normal endocrine islets of Langerhans in number and size as well as normal endocrine acinar tissue. As can be seen with reference to Panel B, a section of the pancreas from normal rat dosed orally with water in addition to an MSM and EDTA composition shows normal endocrine islets of Langerhans in number and size as well as normal endocrine acinar tissue. As can be seen with reference to Panel C, a section of the pancreas from diabetic rat dosed orally with water without an MSM and EDTA composition shows pancreas endocrine islets of Langerhans that are greatly reduced in number and size as well as abnormal endocrine acinar tissue. A substantial amount of the islets were small, shrunk, and inconspicuous. As can be seen with reference to Panel D, a section of the pancreas from diabetic rat dosed orally with water in addition to an MSM and EDTA composition shows distinctly improved endocrine islets of Langerhans in number and size as well as the absence of shrinking of the endocrine acinar tissue.

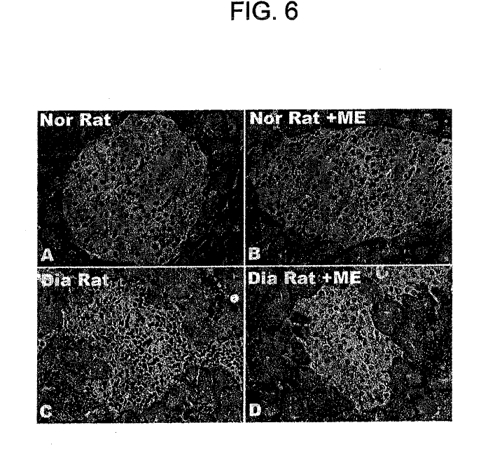

FIG. 6 presents a high magnification (400×) photomicrograph of pancreatic endocrine islets. FIG. 6 shows a 4 μm section of formalin-fixed, paraffin-embedded pancreas that is H&E stained. As can be seen with reference to Panel A, a section of the endocrine islet in the pancreas from normal rat dosed orally with water without an MSM and EDTA composition shows interspersed cells in lightly stained exocrine acinar glands, spherical clusters of cells without ducts, and acini. Panel B presents an endocrine islet in pancreas section from normal rat dosed orally with water and an MSM and EDTA composition, the photomicrograph shows that the histology and morphology were not significantly changed. As can be seen with reference to Panel C, a section of the endocrine islet in the pancreas from diabetic rat dosed orally with water without an MSM and EDTA composition shows that the islets of Langerhans have shrunk and have become small, inconspicuous (e.g., sclerosis of islet and most of the cell's cytoplasm reduced), and also shows the presence of inter-acinar pancreatitis as evident from leukocyte infiltration in the islets. Panel D presents a photomicrograph of an endocrine islet of diabetic rat dosed with an MSM and EDTA composition. The photomicrograph shows that the islets of Langerhans had mild shrinkage and negligible leukocytic infiltration. Accordingly, as presented in FIG. 6, the sections of DR rat pancrease showed inflammatory changes and the reduction in the size and number of islets of Langerhans as compared to the NR rat pancreas, and showed that a composition of MSM and EDTA ameliorated these inflammatory changes.

### Example III

In this experiment 6 to 8 week old Lewis or Sprague Dawley rats with a body weight of 120-140 grams were used to examine the effects of inflammation inside the eye. The role of metal ions on oxidative stress and their relationship to inflammation was studied using an endotoxin-induced uveitis (EIU) model. Acute inflammation was induced in a first group of rats by injecting their hind limb with E coli lipolysaccharide (LPS). A control group was injected with phosphate buffered saline (PBS). Immediately after the injection and every four hours subsequent thereto, one set of rats in the control group and the EIU group was topically treated every 2-4 hours with a composition including EDTA and MSM, wherein the concentration of MSM was at 2.7% and the concentration of EDTA was at 1.25%.

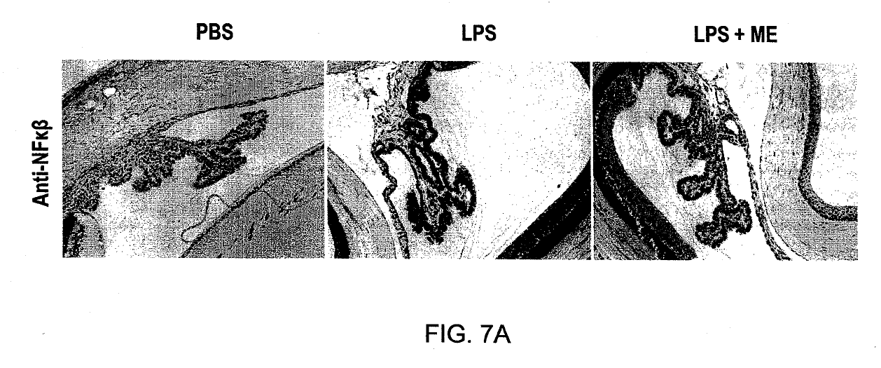

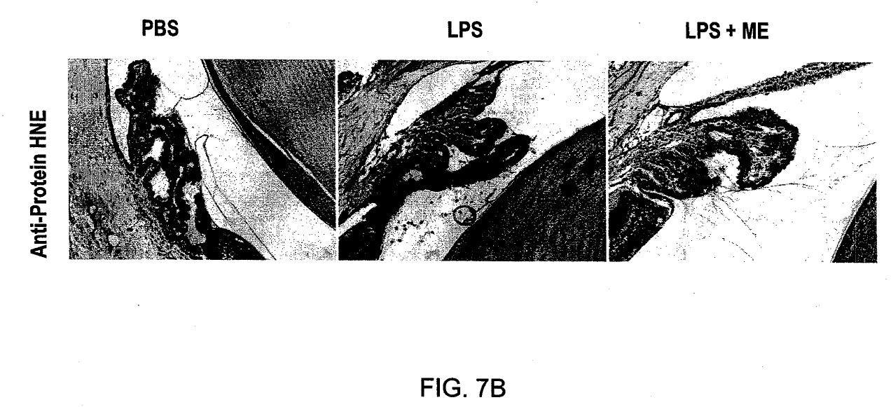

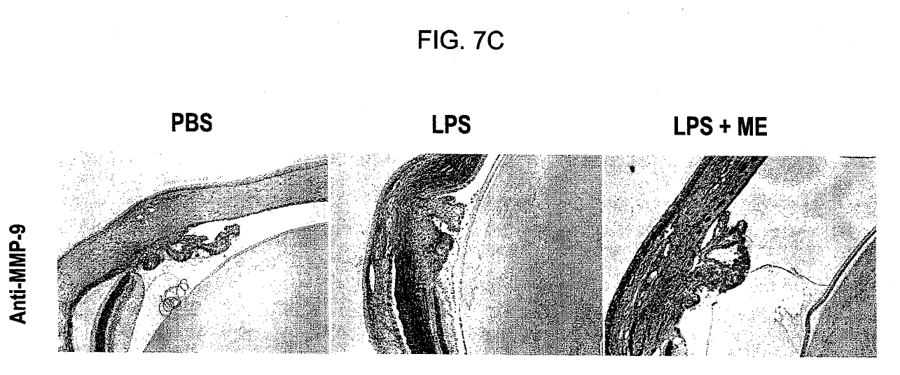

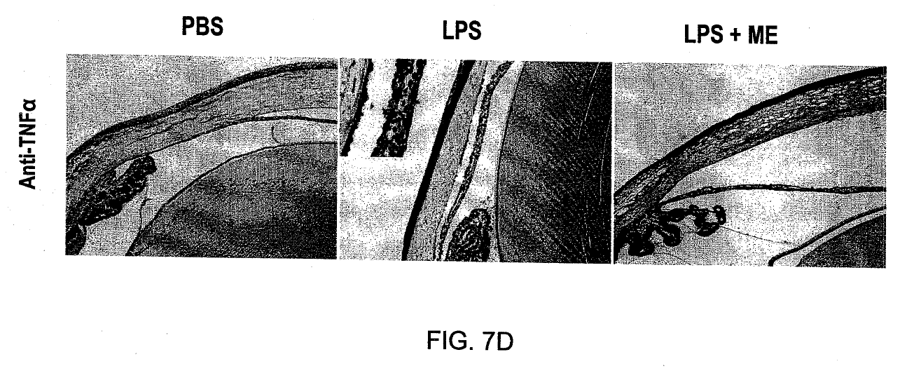

At 6 and 24 hour time points rats were sacrificed, tissue samples obtained, fixed, and immunostained using primary antibodies against NF-κB, protein-HNE, MMP9, and TNF-α (See FIG. 7A-7D). The number of infiltrating cells, proteins, TNF-α, PGE2, and NF-κB, as well as other inflammatory and/or oxidative stress markers were then analyzed in the various tissue sections. At 24 hours, the rats with EIU showed the presence of infiltrating cells, protein, TNF-α, and PGE2, and additionally evidenced a more pronounced NF-κB activation (for instance, at 6 hours). In comparison, the levels of these markers were significantly lower in the EIU rats treated with the EDTA and MSM composition. The control rats showed none of these signs. Immunohistochemistry demonstrated that the increase in inflammatory and oxidative markers in the EIU rats was significantly suppressed by the EDTA and MSM composition. These results indicate that a topical application of an EDTA and MSM composition is effective for inhibiting the activation of NF-κB, MMP-9, and the release of TNF-α, thereby decreasing inflammation.

All publications and patents cited in this specification are herein incorporated by reference as if each individual publication or patent were specifically and individually indicated to be incorporated by reference.

While the disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the disclosure. All such modifications are intended to be within the scope of the claims appended hereto.

1. A method for treating an inflammatory condition in a subject, comprising administering to the subject a therapeutically effective amount of a metal ion sequestering agent and a sequestration inactivating moiety that inactivates the ability of the metal ion sequestering agent to sequester metal ions and facilitates transport of the metal ion sequestering agent across biological membranes, wherein the sequestration inactivating moiety is released in vivo to provide an active metal ion sequestering agent that directly or indirectly exerts an anti-inflammatory effect within the body.

2. The method of claim 1, wherein the metal ion sequestering agent and the sequestration inactivating moiety are administered to the subject in a single composition in which, prior to administration of the composition, the metal ion sequestering agent and the sequestration inactivating moiety are associated so as to inactivate the metal ion sequestering agent.

3. The method of claim 2, wherein the association between the metal ion sequestering agent and the sequestration inactivating moiety comprises covalent attachment.

4. The method of claim 2, wherein the covalent attachment is severed by a chemical reaction in vivo.

5. The method of claim 4, wherein the chemical reaction is enzymatic.

6. The method of claim 4, wherein the chemical reaction is nonenzymatic.

7. The method of claim 1, wherein the sequestration inactivating moiety comprises a metal ion sequestered by the metal ion sequestering agent, said metal ion being displaceable in vivo.

8. The method of claim 1, wherein the sequestration inactivating moiety ionically binds to at least one coordinating atom in the metal ion sequestering agent.

9. The method of claim 1, wherein the at least one coordinating atom is a nitrogen atom and the sequestration inactivating moiety is anionic.

10. The method of claim 9, wherein the at least one coordinating atom is an oxygen atom and the sequestration inactivating moiety is cationic.

11. The method of claim 1, wherein the sequestration inactivating moiety hydrogen bonds to at least one coordinating atom in the metal ion sequestering agent.

12. The method of claim 2, wherein the composition consists essentially of the metal ion sequestering agent and the sequestration inactivating moiety.

13. The method of claim 2, wherein the composition further comprises an anti-inflammatory agent.

14. The method of claim 13, wherein the composition consists essentially of the metal ion sequestering agent, the sequestration inactivating moiety, and the anti-inflammatory agent.

15. The method of claim 1, wherein the metal ion sequestering agent is an iron chelator.

16. The method of claim 1, wherein the metal ion sequestering agent is a calcium chelator.

17. The method of claim 1, wherein the metal ion sequestering agent is a magnesium chelator.

18. The method of claim 1, wherein the metal ion sequestering agent is ethylenediamine tetraacetic acid (EDTA) or a pharmacologically acceptable salt thereof, and the sequestration inactivating moiety is methysulfonylmethane.

19. The method of claim 15, wherein the molar ratio of the MSM to the EDTA in the composition is in the range of about 4:1 to about 10:1.

20. The method of claim 19, wherein the molar ratio of the MSM to the EDTA in the composition is in the range of about 6:1 to about 8:1.

21. The method of claim 1, wherein the inflammatory condition is selected from hypersensitivities, immune and autoimmune disorders, gastrointestinal diseases, cancer, vascular complications, heart conditions, liver conditions, kidney conditions, neurodegenerative conditions, pelvic inflammatory disorders, ulcers, ulcer-related disorders, age-related disorders, preeclampsia, conditions related to chemically induced, radiation-induced, or thermally induced physical trauma, acute inflammatory conditions, and chronic inflammatory conditions.

22. The method of claim 1, wherein the composition is administered to the subject via a route of administration that is other than ophthalmic.

23. The method of claim 22, wherein the composition is systemically administered to the subject.

24. A composition for the treatment of an inflammatory condition, comprising a therapeutically effective amount of an anti-inflammatory agent, a therapeutically effective amount of a metal ion sequestering agent, and a sequestration inactivating moiety that inactivates the ability of the metal ion sequestering agent to sequester metal ions and facilitates the transport of the metal ion sequestering agent through biological membranes, wherein the sequestration inactivating moiety is released in vivo to provide an active metal ion sequestering agent that directly or indirectly exerts an anti-inflammatory effect within the body.

25. The composition of claim 24, wherein the metal ion sequestering agent and the sequestration inactivating moiety are associated so as to inactivate the metal ion sequestering agent.

26. The formulation of claim 25, wherein the association between the metal ion sequestering agent and the sequestration inactivating moiety comprises covalent attachment.

27. The formulation of claim 26, wherein the covalent attachment is severed by a chemical reaction in vivo.

28. The formulation of claim 27, wherein the chemical reaction is enzymatic.

29. The formulation of claim 27, wherein the chemical reaction is nonenzymatic.

30. The formulation of claim 24, wherein the sequestration inactivating moiety comprises a metal ion sequestered by the metal ion sequestering agent, said metal ion being displaceable in vivo.

31. The formulation of claim 24, wherein the sequestration inactivating moiety ionically binds to at least one coordinating atom in the metal ion sequestering agent.

32. The formulation of claim 31, wherein the at least one coordinating atom is a nitrogen atom and the sequestration inactivating moiety is anionic.

33. The formulation of claim 31, wherein the at least one coordinating atom is an oxygen atom and the sequestration inactivating moiety is cationic.

34. The formulation of claim 24, wherein the sequestration inactivating moiety hydrogen bonds to at least one coordinating atom in the metal ion sequestering agent.

35. The formulation of claim 24, wherein the formulation consists essentially of the anti-inflammatory agent, the metal ion sequestering agent, and the sequestration inactivating moiety.

36. The formulation of claim 24, wherein the metal ion sequestering agent is an iron chelator.

37. The formulation of claim 24, wherein the metal ion sequestering agent is a calcium chelator.

38. The formulation of claim 24, wherein the metal ion sequestering agent is a magnesium chelator.

39. The formulation of claim 24, wherein the metal ion sequestering agent is ethylenediamine tetraacetic acid (EDTA) or a pharmacologically acceptable salt thereof, and the sequestration inactivating moiety is methysulfonylmethane.

40. The formulation of claim 34, wherein the molar ratio of the MSM to the EDTA in the formulation is in the range of about 4:1 to about 10:1.

41. The formulation of claim 40, wherein the molar ratio of the MSM to the EDTA in the formulation is in the range of about 6:1 to about 8:1.

42. A composition consisting essentially of a therapeutically effective amount of a metal ion sequestering agent and a sequestration inactivating moiety that is effective to facilitate transport of the metal ion sequestering agent through biological membranes, wherein the amount of the sequestration inactivating moiety in the composition is sufficient to inactivate the inability of the metal ion sequestering agent to sequester metal ions until the sequestration inactivating moiety is released in vivo to provide an active metal ion sequestering agent that directly or indirectly exerts an anti-inflammatory effect within the body.
